(19) United States
(12) Patent Application Publication
(10) Pub. No.: US 2003/0186248 A1
(43) Pub. Date: Oct. 2, 2003

(54) Interpreting cytological specimens via molecular histological signatures

(76) Inventors: Mark G. Erlander, Encinitas, CA (US); Dennis C. Sgroi, Winchester, MA (US); Xiao-Jun Ma, San Diego, CA (US); Thomas M. Baer, Mountain View, CA (US)

Correspondence Address: Kawai Lau, Morrison & Foerster LLP, Suite 500, 3811 Valley Centre Drive, San Diego, CA, 92130-2332, US

(21) Appl. No.: 10/109,953

(22) Filed: Mar. 29, 2002

(51) Int. Cl. C12Q 1/68; G01N 1/30; G01N 33/48

(52) U.S. Cl. 435/6; 435/40.5

(57) ABSTRACT

The present invention relates to the use of molecular histological signatures to interpret and correlate cytological specimens with the presence or absence of disease and the progression thereof. The invention provides molecular signatures for use in the study and/or diagnosis of diseased cells and tissues of a cytological specimen relative to a solid (e.g. histological) sample.

### FIELD OF THE INVENTION

The invention relates to the use of molecular histological signatures to interpret and correlate cytological specimens with the presence or absence of disease as well as the extent of disease progression when a disease is present. Molecular signatures, embodied in nucleic acid expression and/or protein expression or other formats, are used in the study and/or diagnosis of diseased cells and tissues of a cytological specimen relative to a solid (e.g. histological) sample. The signatures represent an advance in molecular medicine and may also be used in the study and/or determination of disease subtypes, treatment methods and the prognosis of a patient.

### BACKGROUND OF THE INVENTION

Disease treatment begins with diagnosis. Diagnosis is often performed in whole or in part by a pathologist, who interprets the morphology of cell and/or tissue sample from a subject to determine the presence or absence of disease and/or disease stage which leads to a determination of the recommended therapy for the disease. The determination of disease presence involves the risks of (1) incorrectly determining the presence of disease when it is absent and (2) incorrectly determining the absence of disease when it is present. The first risk is that of a “false positive” which may result in an unnecessary (and often painful, disfiguring, and/or costly) treatment procedure. The second risk is that of a “false negative” which may result in the non-detection of a life threatening condition.

The determination of disease stage is equally critical because clinical treatment modalities are often different depending on disease progression. Thus once again, there are the risks of “false positives”, which may result in the application of an unnecessary procedure, and “false negatives”, which may result in the non-application of a necessary procedure to prolong life.

To reduce the risks noted above, pathologists obtain samples to assist in achieving the correct diagnosis. Pathology samples may be broadly divided into three types: whole tissue samples, cytological samples, and blood samples. Whole tissue samples normally require some type of surgical or invasive procedure and may be further divided into bulk tissue samples, histology samples (frozen or fixed/embedded), cultured samples, and flow cytometry sorted samples. Histology samples provide the advantageous ability to evaluate cells and tissues “in situ” such that the context of the cells in the tissue and the characteristics of surrounding regions can provide insight beyond the cytomorphology of the cell and its contents to assist in determining disease presence and/or disease progression. This is in contrast to bulk tissue and flow cytometry samples which provide little or no information by an “in situ” context. Cultured samples present a problem in that the relationship between cells in culture and in vivo has not been established.

Cytological samples or specimens are of two basic types. The first utilizes either spontaneous or abraded (forcibly removed) exfoliates. Examples of the former are nipple secretions, vaginal fluids, cerebrospinal fluid, urine, or serrous effusions. Examples of the latter are ductal lavage, cervical smears, or other washings or brushings. The second type of cytological specimen is obtained by fine needle aspiration (FNA) biopsy. Both types may be viewed as being collected through non-invasive or minimally invasive techniques which are readily performed in a clinical setting. They are more attractive than that of a surgical procedure to obtain solid tissue samples, which often require a painful procedure, radiology for visualization, possible deformity, and increased costs. The samples provide, however, no “in situ” context of the cells because much, if not all, of the in vivo histological architectural patterns and histopathology is lost with the removal of cells from the subject. Additionally, the small size of aspirated specimens do not allow for ancillary tests or limits the number of studies that can be performed on the specimen. Thus, the correlation between cytomorphology and disease is more difficult for cytological specimens than for solid samples. The limitations of cytological specimens often leads to a requirement for a histology sample as described above.

Blood samples provide no in vivo architecture, leaving little beyond the cytomorphology of cells in the sample to assist a pathologist. Except for bloodborne cells, blood is also less likely to contain disease cells unless they are of a type that would exfoliate into the bloodstream, such as those of metastatic cancer as opposed to a primary tumor.

Given the advantages and disadvantages of cytological specimens in comparison to histological samples as noted above, it has become a goal to augment or otherwise improve cytological sampling by correlating its analysis with that of solid histological samples. In breast cancer, for example, cytological specimens are often classified as one of the following: insufficient sample size, benign (with various proliferative types therein), a typical, suspicious, and malignant. This is in contrast to a breast cancer histological sample which can be examined by a trained pathologist to determine whether ductal epithelial cells are normal (e.g. not precancerous or cancerous or having another noncancerous abnormality), precancerous (e.g. comprising hyperplasia such as a typical ductal hyperplasia (ADH)) or cancerous (comprising ductal carcinoma in situ, or DCIS, which includes low grade ductal carcinoma in situ, or LGDCIS, and high grade ductal carcinoma in situ, or HG-DCIS) or invasive (ductal) carcinoma (which includes low grade invasive ductal carcinoma, or LG-IDC, high grade invasive ductal carcinoma, or HG-IDC, and intermediate grades of IDC). Pathologists may also identify the occurrence of lobular carcinoma in situ (LCIS) or invasive lobular carcinoma (ILC). An “invasive” carcinoma can invade and damage nearby tissues and organs as well as metastasize, entering the bloodstream or lymphatic system. Breast cancer progression may be viewed as the occurrence of abnormal cells, such as those of ADH, DCIS, IDC, LCIS, and/or ILC, among normal cells.

Importantly, cytological specimens cannot differentiate between a typical ductal hyperplasia from carcinomas. This has important implications because it remains unclear whether normal cells become a typical (such as ADH) and then progress on to become malignant (DCIS, IDC, LCIS, and/or ILC) or whether normal cells are able to directly become malignant without transitioning through an a typical stage. It has been observed via prospective trials, however, that the presence of ADH indicates a higher likelihood of developing a malignancy. This has resulted in treatment of patients with ADH with an antiestrogen/antitumor agent such as tamoxifen. This is in contrast to the treatment of patients with malignant breast cancer which usually includes surgical removal.

Cytological specimens also cannot differentiate between in situ and invasive ductal carcinomas. Thus at least the cytological specimens identified as a typical or suspicious (and thus possibly indicative of hyperplasia or carcinoma) or malignant (and thus indicative of in situ or invasive carcinoma) are likely to require an additional histological sampling to improve the determination of whether, or what type of, carcinoma is present. The inability to differentiate between in situ and invasive ductal carcinomas remains despite the availability of a few molecular alterations that have been identified as correlated with breast tumors. These alterations include the presence or absence of the estrogen and progesterone steroid receptors, gross cystic disease fluid protein (GCDFP), and 15/AP-15. Other molecular alterations that have been reported in breast cancer include HER-2 expression/amplification (Mark H F, et al. Genet Med; 1(3):98-103 1999), Ki-67 (an antigen that is present in all stages of the cell cycle except G0 and used as a marker for tumor cell proliferation, and prognostic markers (including oncogenes, tumor suppressor genes, and angiogenesis markers) like p53, p27, Cathepsin D, pS2, multi-drug resistance (MDR) gene, and CD31.

The usefulness of cytological specimens in reducing the need for invasive histological sampling in breast cancer and other diseases would be greatly enhanced by the identification of molecular alterations correlated with the presence of cancer and/or its various stages. Unfortunately, relatively little is known of such alterations despite intense study. The use of cDNA libraries to analyze differences in gene expression patterns in normal versus tumorigenic cells has been described (U.S. Pat. No. 4,981,783). DeRisi et al. (1996) describe the analysis of gene expression patterns between two cell lines: UACC-903, which is a tumorigenic human melanoma cell line, and UACC-903(+6), which is a chromosome 6 suppressed non-tumorigenic form of UACC-903. Labeled cDNA probes made from mRNA from these cell lines were applied to DNA microarrays containing 870 different cDNAs and controls. Genes that were preferentially expressed in one of the two cell lines were identified.

Golub et al. (1999) describe the use of gene expression monitoring as means to cancer class discovery and class prediction between acute myeloid leukemia (AML) and acute lymphoblastic leukemia (ALL). Their approach to class predictors used a nearest neighbor analysis followed by cross-validation of the validity of the predictors by withholding one sample and building a predictor based only on the remaining samples. This predictor is then used to predict the class of the withheld sample. They also used cluster analysis to identify new classes (or subtypes) within the AML and ALL.

Gene expression patterns in human breast cancers have been described by Perou et al. (1999), who studied gene expression between cultured human mammary epithelia cells (HMEC) and breast tissue samples by use of microarrays comprising about 5000 genes. They used a clustering algorithm to identify patterns of expression in HMEC and tissue samples. Perou et al. (2000) describe the use of clustered gene expression patterns to classify subtypes of human breast tumors. Hedenfalk et al. describe gene expression patterns in BRCA1 mutation positive, BRCA2 mutation positive, and sporadic tumors. Sgroi et al. also analyzed gene expression patterns of normal and breast cancer cells from a single patient. Using gene expression patterns to distinguish breast tumor subclasses and predict clinical implications is described by Sorlie et al. and West et al.

None of the above described approaches, however, relate the gene expression profile of a cytological specimen with a diagnosis based upon the molecular histological signature of a solid histological sample from a patient with a disease. No genetic alterations have been identified in the art to distinguish the pathological stages of breast cancer (e.g. ADH, LG-DCIS, HG-DCIS, LG-DCIS, LG-IDC, and HG-IDC) or the pathological grades (i.e. grades I, II, and III) of DCIS and IDC.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present invention relates to the correlation of the molecular signature of one or more cells of a cytological specimen with the phenotype of one or more cells of a histological sample. Such methods of correlating may be accomplished by comparing the molecular signature of the cell(s) of a cytological specimen with the molecular signature of cells corresponding to a particular phenotype. Equivalence between the two signatures indicates that the cell(s) of the specimen have the phenotype of the sample.

In one embodiment of the invention, the molecular signature corresponding to a phenotype of the histological sample may have been previously determined or identified and available as a reference to which the molecular signature of the cell(s) of a cytological specimen may be correlated or compared. As used herein, “phenotype” refers to the manifestation of effects (or results) from the expression of one or more biomolecules, including effects (or results) at the cellular, tissue, system, and/or organism level. A difference in phenotype between two cells does not necessarily reflect a difference in genotype, although a different genotype may be involved in certain instances, such as, but not limited to, amplified genomic material (e.g. gene amplification), mutated genetic material (e.g. gene mutation in a cell), or exogenous genetic material (e.g. viral infection).

In one application of the invention, one or more cells of a cytological specimen from a subject is used to identify and/or diagnose a phenotype, such as the presence and/or stage of a disease in said cell(s), by reference to the molecular signature of a histological sample corresponding to the phenotype. Preferably, the invention is practiced without obtaining an actual histological sample from said subject to identify and/or diagnose the phenotype (such as by use of “reference” signature as discussed below. The molecular signature of the cells of a “reference” histological sample may be compared to the molecular signature of the cell(s) of a cytological specimen. Stated differently, the invention provides the ability to identify and/or diagnose a cytological specimen by comparing the molecular signature of one or more cells of the specimen with the molecular signature of cells of an identified or diagnosed histological sample. “Reference” signatures of any histological sample can be prepared and used in the practice of the present invention. The “reference” signatures of the invention may be in the form of a database which is optionally in electronic form. Such a database may contain each “reference” signature individually and/or a composite signature, or “model” based upon all or part of the individual signatures.

It is possible, however, to also obtain a histological sample from the subject, from whom one or more cytological specimens are obtained, for the preparation of reference molecular signature(s) for comparison to signature(s) of cell(s) of the specimen(s).

The present invention may be applied in relation to any phenotype, but in preferred embodiments it is applied with respect to a disease condition wherein cells of a subject have aberrant or altered gene expression (including responses to infection such as by bacteria, mycobacteria and fungi) and may be collected by cytological means. Non-limiting examples include cancer, viral infection, autoimmune diseases, arthritis, diabetes and other metabolic diseases. Cytologically collected specimens refers to samples removed by non-invasive or minimally invasive means from a subject afflicted with, or suspected of being afflicted with, the disease condition. In an alternative embodiment of the invention, the methods may also be practiced with cytological specimens collected from the population at large for population screening to identify a typical or malignant cells or other cells of clinical relevance.

Preferred cytological specimens are either spontaneous or abraded exfoliates or fine needle aspirates obtained via a biopsy procedure. Particularly preferred are specimens collected via a PAP smear, ductal lavage, fine needle aspiration, prostate massage, sputum (including saliva, bronchial brush or bronchial wash), stool, semen, urine, or other bodily fluid (including ascitic fluid, cerebral spinal fluid (CSF), bladder wash, and pleural fluid). Non-limiting examples of tissues susceptible to fine needle aspiration include lymph node, lung, thyroid, breast, and liver.

Cytological specimens may be prepared for use in the present invention by a variety of ways known in the art, including, but not limited to, concentration of cells in the specimen, mounting or fixation on a solid support such as a slide, cover slide, and staining of cells in the specimen. The stains may be histochemical or immunochemical in nature as known in the art and discussed herein. One or more cells of the prepared specimen are isolated from the cytological specimen and used to prepare a molecular signature of said cell(s). In preferred embodiments of the invention, the isolation of one or more cells is performed by microdissection, such as, but not limited to, laser capture microdissection (LCM) or laser microdissection (LMD). Alternatively, the invention may be practiced without isolation of cells such that the cytological specimen is used directly to prepare a molecular signature. The molecular signature is reflective of the levels and/or activities of one or more biomolecules that are present and assayable from the cells of the cytological sample. The biomolecule(s) may be any that are found in the cells, but are RNA (e.g. mRNA), DNA or protein molecules in preferred embodiments of the invention. The levels and/or activities of the biomolecule(s) may be assayed directly or indirectly, or may be amplified in whole or in part prior to detection.

The molecular signature prepared from the cytological specimen is then compared with the molecular signature of cells of a solid tissue (histological) sample which have been identified as being those of a particular phenotype, such as, but not limited to, a disease type and/or stage of a disease and/or a sensitivity or resistance to a particular therapy or treatment. The molecular signatures of cells of a solid histological sample are thus “reference” histological signatures with which the signatures of cytological specimens are compared. Such “reference” signatures may correspond to any phenotype of normal or benign cells found in the sample as well as disease afflicted cells found in the sample.

The identification of cells in a solid histological sample as having a phenotype such as, but not limited to, being normal or benign, or corresponding to a particular disease or disease stage, may be performed by a skilled pathologist using known techniques, including the use of cytomorphological information not available in cytological specimens, to distinguish between normal cells and disease afflicted cells as well as the progression of the disease in afflicted cells. The cell(s) identified as being one or more phenotypes are isolated and used to prepare molecular signatures reflecting the levels and/or activities of one or more biomolecules that are present and assayable from the cell(s). The isolation of one or more cells from a solid histological sample may be performed by any means, but is preferably performed by microdissection, such as, but not limited to, laser capture microdissection, after staining. The isolation of cells advantageously permits the exclusion of unrelated cell types such as, but not limited to, infiltrating immune cells, as well as exclusion of cells of other phenotype(s). The preparation of the molecular signature is preferably by the same means as that used to prepare the molecular signature of the cytological specimen.

The comparison of a molecular signature from a cytological specimen with one or more “reference” histological signatures may be an assessment of the relative change in level of or presence/absence of a single biomolecule. Stated differently, the comparison may be quantitative or qualitative. In this embodiment of the invention, each “signature” is the expression or activity of a single biomolecule. Alternatively, the comparison may be an assessment of quantitative or qualitative changes in multiple biomolecules. In this embodiment, each “signature” is the expression or activity or more than one biomolecule. A “signature” of a single biomolecule may be used with significant accuracy although a “signature” of multiple biomolecules may increase the ability to accurately discriminate between the presence/absence of a phenotype, such as a disease condition, or between various phenotypes, such as stages of a disease. The presence of a corresponding, comparable, equivalent, same, matching, or identical molecular signature between a cytological specimen and a histological sample identifies the cells of the cytological specimen as having the same phenotype as those of the histological sample. Applied to diseases, the presence of the same molecular signature is indicative of cells of the cytological specimen as having the normal, benign, or diseased phenotype of a histological sample. It should be noted, however, that identity between signatures is not necessary; a positive correlation between the two signatures is sufficient.

In addition to comparisons with “reference” histological signatures of different disease stages, the present invention provides for comparisons of molecular signatures of cytological specimens with “reference” histological signatures of different subtypes of a disease condition as phenotypes. Non-limiting examples include various subtypes of “benign” conditions as well as various subtypes of a stage (such as, but not limited to, various “grades” of an invasive carcinoma like that seen in breast cancer). A skilled pathologist, using techniques known in the art, can readily assist with this aspect of the invention by identifying one or more cells of a solid histological sample as being those of various subtypes of a disease condition. The cell(s) are then isolated by subtype and used to prepare “reference” histological signatures of individual subtypes. The presence of the same signature between a cytological specimen and a subtype histological sample identifies the cells of the cytological specimen as of the same subtype as the sample.

The present invention further provides for comparisons of molecular signatures of cytological specimens with histological signatures of disease prognosis or outcome phenotypes at the cell, tissue, system, and/or organism level as observed in subjects with cells having the signature in a histological sample. Non-limiting examples of such phenotypes include mortality rates, life expectancy under various conditions, and sensitivity or resistance to a particular therapeutic agent or treatment, including information regarding the likelihood of success or failure of various treatment regimens for the disease. The histological signatures corresponding to such phenotypes may be readily identified by correlating various “reference” signatures to the subsequent treatments and outcomes observed for “reference” subjects having said signatures. One means of correlating is by comparison to prospective studies of various diseases or disease treatments. Signatures that correlate with particular outcomes or sensitivities/resistance are then identified as “reference” histological signatures of individual phenotypes. The presence of the same signature between a cytological specimen and a histological sample identifies the subject, such as a patient, as having the same phenotype as the “reference” subject.

The present invention thus provides means for correlating a molecular expression pattern with a physiological condition, such as the state of a disease, and/or prognosis, including possible or likely outcomes under various treatments. This correlation provides a way to molecularly diagnose and/or monitor the status of a cell or a patient in comparison to different diseased versus non-diseased phenotypes as discussed herein.

The ability to diagnose is provided by the identification of expression of the individual biomolecules as relevant for the determining a phenotype such as the presence and/or stage or subtype of a disease condition. The invention is not limited by the form of the assay used to determine the presence or level of expression of a biomolecule. An assay may utilize any identifying feature of an identified biomolecule as disclosed herein as long as the assay reflects, quantitatively or qualitatively, expression of the biomolecule. Identifying features include, but are not limited to, unique nucleic acid sequences used to encode (e.g. DNA), or express (e.g. RNA or protein), said biomolecule (e.g. cellular components) or epitopes specific to, or activities of, the biomolecule. Other identifying features include the physical form of cellular components, including, but not limited to, the modification (e.g. methylation) of nucleic acid sequences used to encode a biomolecule as well as modifications of the biomolecule itself (e.g. of a protein by phosphorylation, glycosylation, proteolytic cleavage, etc.). The invention may also be practiced with the use of one or more single nucleotide polymorphisms as an identifying feature of a biomolecule. The invention simply utilizes the identity of the biomolecule(s) necessary to identify the presence of, or to discriminate between, phenotypes (e.g. a disease condition).

The invention also provides for the identification of individual “reference” histological signatures corresponding to various phenotypes by analyzing global, or near global, biomolecule expression from single cells or homogenous cell populations (of a solid histological sample) which have been dissected away from, or otherwise isolated or purified from, contaminating cells beyond that possible by a simple biopsy. Because the expression of numerous biomolecules vary between cells from different patients as well as between cells from the same patient sample, multiple individual biomolecule expression patterns are used as reference data to generate models of expression to be used as the basis of “reference” signatures. Individual expression patterns of cells of a phenotype are compared to identify biomolecule(s) the expression (or non-expression) of which are most highly correlated with the phenotype (e.g. relating to a disease or disease phenotype such as disease stage or subtype). Comparisons of large amounts of “reference” signature data improve the correlation between a model based upon the detected expression(s), and/or non-expression(s), and the phenotype identified with the model. A “reference” signature based upon such a model is preferably present in all samples used to generate the model, and is preferred in the practice of the invention. Such signatures are likely to have the best ability to discriminate cells of one disease, stage or subtype from another.

The invention also provides for the use of molecular signatures found in cells of cytological specimens as correlating to various phenotypes or for modifying, refining, or improving models of expression based upon histological signatures. The molecular signatures of cells in cytological specimens may also be used as reference data to inform a “model” signature. Preferably, such use occurs after the cytological specimen is confirmed by independent means as having the phenotype as that of the “model” signature.

In another aspect of the invention, the molecular signatures from histological samples may be used to identify the molecular signatures of one or more subsets of the samples. Such “subset” signatures correspond to a “subphenotype” of the phenotype of the histological samples. Preferably, the samples are from more than one subject identified as having the same phenotype such that the molecular signatures of said samples may be analyzed to identify one or more biomolecule(s) the expression (or non-expression) of which are most highly correlated with a subset (i.e. less than all) of the samples. A “reference” molecular signature based upon the detected expression(s), and/or non-expression(s), is thus indicative of the subset (and thus subphenotype) when present in a cytological specimen. The phenotype of the subset (i.e. subphenotype) may be further characterized by correlating the molecular signatures of these subsets with observations at the cell, tissue, system, and/or organism level of the subject in which the subset is or was present. Applied to a disease subset as a non-limiting example, observations over the course of the disease (in the subjects or patients from whom the samples were taken) are correlated with the subset to identify additional characteristics of the subset. Examples of additional characteristics include disease outcomes or responses to various treatments. Observations made after the isolation of the samples from the subjects may also be used. The molecular signatures of subsets may of course also be included as part of a reference database and/or to modify other “reference” signatures as disclosed herein. Comparison of the molecular signature of a subset and the molecular signature of a cytological specimen may be used to identify the specimen as having the same phenotype as the subset.

In an alternative embodiment of the invention, the molecular signatures from cytological specimens may also be to identify the molecular signatures of one or more subsets of the specimens. This may be done by comparison to subset signatures of histological samples as discussed above. Alternatively, this may be done by comparison to “reference” signatures from histological samples of more than one subject identified as having the same phenotype such that the molecular signatures of said specimens may be analyzed to identify one or more biomolecule(s) the expression (or non-expression) of which are most highly correlated with a subset (i.e. less than all) of the specimens (as well as the histological samples). A molecular signature based upon the detected expression(s), and/or non-expression(s), is thus indicative of the subset (and thus a subphenotype) when present in a cytological specimen (or in a histological sample). As described above, the phenotype of the subset (i.e. subphenotype) may be further characterized by correlation with observations at the cell, tissue, system, and/or organism level of the subject in which the subset is or was present. In the case of subsets of a disease, observations over the course of the disease (in the subjects or patients from whom the specimens, or samples, were taken), such as disease outcomes or responses to various treatments, are correlated with the subset to identify additional characteristics of the subset. Observations made after the isolation of the samples from the subjects may also be used.

In embodiments of the invention for detecting the presence of a disease condition as a phenotype, the invention provides for the comparison of a molecular signature of a cytological specimen to a “reference” histological signature of a solid histological sample. A cytological specimen is obtained from a subject suspected of being afflicted with a disease and analyzed for the presence of one or more cells suspected of being indicative of, or involved in the progression of, the disease. These cell(s) are then isolated and the molecular signature of one or more expressed biomolecules prepared. Alternatively, the cytological specimen is utilized in toto, without the need for analysis to identify suspect cells, to prepare a molecular signature. In another alternate embodiment, the cytological specimen is obtained from a subject in the general population to screen for the presence of disease in the subject. This may be performed as part of a routine health “check up” and is analogous to screening procedures such as mammography or PSA tests. Of course the presence of cells indicative of disease or involved in disease progression may also be used to detect the presence of the disease. The present invention, however, provides an advance by allowing the identification of particular disease related phenotypes.

The molecular signature of a cytological specimen is compared to known molecular signatures of cells of a histological sample that have been identified or diagnosed as being of said disease condition to determine whether the specimen contains the presence of the disease. Stated differently, comparison of a molecular signature of a cytological specimen to a “reference” histological signature of a solid histological sample is used for identifying and/or diagnosing particular stages and/or subtypes of a disease. Using breast cancer as a exemplary and non-limiting example of the present invention, cells from a cytological specimen are stained (e.g. with the stain used for PAP smears) examined for those that appear “a typical” or “suspicious” and suspected of being cancer related. The cells are isolated, and a molecular signature of prepared and compared to a “reference” signature of a histological sample known to be cancerous to determine whether the cells are cancerous. Alternatively, the cells may be identified as ADH, in which case the afflicted subject may be directed to begin treatment with an antiestrogen/antitumor agent such as tamoxifen. This is in contrast to the treatment of patients with malignant breast cancer which usually includes surgical removal.

In an alternative embodiment of the invention, a fine needle aspirate (FNA) of a lump in a subject having or suspected of having breast cancer may be used as a cytological specimen that is used in whole or in part to prepare a molecular signature without the selection of cells suspected of cancer related. Such FNA specimens often contain large numbers of breast cancer cells, and the molecular signature of the specimen needs only be compared with “reference” signatures to detect the presence of the signature corresponding to the highest grade (or stage) of cancer the specimen to assist in the diagnosis and determination of subsequent treatment.

From a cytological specimen, cell(s) are isolated and the molecular signature of one or more expressed biomolecules prepared. This molecular signature is then compared to known molecular signatures of cells of a histological sample that have been identified or diagnosed as being of a stage or subtype of said disease condition to determine whether the cytological specimen contains the presence of the same stage or subtype of the disease. Using breast cancer as an exemplary and non-limiting example of the application of the present invention, cells from a cytological specimen are examined for those that appear “malignant”. The cells are isolated, and a molecular signature of prepared and compared to a “reference” signature of a histological sample known to be that of DCIS or IDC and/or various grades (“low” versus “intermediate” or “high” or “I, II, or III”) thereof. Similarly, cells that appear “benign” may be isolated and used to prepare a molecular signature to determine what level of continued risk, if any, that they pose to the patient by comparison to the outcomes seen in previous patients having the same histological signature.

The present invention may also be advantageously applied to the identification of recommended therapeutic treatments and/or the determination of prognosis based upon the observed molecular signature of a cytological specimen in comparison with “reference” molecular histological signatures of various phenotypes of diseases, disease stages, and disease subtypes. By evaluating the signature of a patient's cytological specimen in relation to “reference” signatures for which a preferred course of therapy or prospective knowledge concerning patient outcome is known, decisions concerning treatment of the patient may be modified or determined based upon the disease, stage and/or subtype identified. This has already been noted above with respect to the identification of ADH in a cytological specimen. This aspect of the invention may also be applied, however, to the determination of appropriate treatments for DCIS versus IDC patients as well as of the various grades of these types of malignancies.

Exemplary embodiments of the above aspects of the invention comprise one or more of the followed preferred means of practicing the invention: the cytological specimen is collected by non-invasive or minimally invasive means (such as an exfoliate or fine needle aspirate); the cell(s) of the specimen are isolated by microdissection after staining as deemed appropriate or necessary; the molecular signature is that of more than one expressed biomolecule and is prepared by amplification of expressed nucleic acid sequences; the cells of the histological sample are collected via microdissection; the molecular signature of cells of the histological sample is that of more than one expressed biomolecule and is prepared by amplification of expressed nucleic acid sequences; and/or molecular signatures are embodied in arrays or “microarrays” of known nucleic acid molecules hybridized to nucleic acids amplified from isolated cells of a cytological specimen.

Use of microdissection is a preferred aspect of the invention because contaminating, non-disease related cells (such as infiltrating lymphocytes or other immune system cells) may be eliminated from a cytological specimen or histological sample to avoid the possibility of affecting the biomolecules identified or the subsequent analysis thereof to identify the status of suspect cells. Such contamination is present where a biopsy is used to generate a gene expression profile as a “reference” signature without further isolation of cancer related cells (such as by microdissection). Contamination may also be obviated by use of a molecular signature that is not affected by contaminating, non-disease cells, such as via detection of expression of one or more biomolecules that are not expressed in contaminating cells.

The present invention also offers the benefit of reducing the occurrence of false negative diagnoses by permitting diagnosis (via a molecular signature) based on a few malignant cells (which would otherwise be insufficient for diagnosis based upon cytological methods alone) and by lowering the likelihood of screening or interpretive errors that may occur based upon cytological methods alone.

While the present invention is described further below in the context of human breast cancer, it may be practiced in the context of any cancer or other disease of any animal. Preferred animals for the application of the present invention are mammals, particularly those important to agricultural applications (such as, but not limited to, cattle, sheep, horses, and other “farm animals”) and for human companionship (such as, but not limited to, dogs and cats). MODES OF CARRYING OUT THE INVENTION

One non-exclusive embodiment of the invention is the use of a molecular signature from a cytological specimen of a subject afflicted with, or suspected of being afflicted with, cancer. The molecular signature of the specimen may be compared to known “reference” molecular histological signatures (also known as “gene expression patterns” or “gene expression profiles”) of cells known to have one or more phenotypes of a particular cancer and/or various stages or subtypes thereof. The cancer may be that of any known type, but preferred are cancers that are susceptible to isolation from a solid histological sample and cytomorphological analysis. The signature of a cytological specimen may also be used for determining diagnosis, therapy and prognosis of the cancer as described herein.

The present invention also provides for the use of a signature of a cytological specimen in the determination of cancer stage and/or subtype. A molecular signature of a cytological specimen is thus correlated with, and able to discriminate between,

(1) pathological stages of cancer (e.g. benign, ADH, DCIS, IDC, and metastatic in breast cancer as non-limiting examples);

(2) pathological grades (e.g. grades I, II, and III of IDC or DCIS in breast cancer as non-limiting examples);

(3) subtypes of a particular cancer (e.g. estrogen receptor positive or negative and Her-2/neu positive or negative in breast cancer as non-limiting examples);

(4) nodal status (quantitative or qualitative);

(5) metastatic potential, especially in node negative patients;

(6) responsiveness or lack thereof to a given therapeutic agent or treatment (e.g. tamoxifen, aromatase inhibitors, or taxol in breast cancer as non-limiting examples); and

(7) aggressiveness of a cancer.

Although not expressly stated, items (2), (4), (5), (6), and (7) also define “subtypes” as described herein.

More broadly defined, the stages are non-malignant versus malignant, but may also be viewed as normal (or benign) versus a typical (optionally including reactive and pre-neoplastic) versus cancerous. Another definition of the stages is normal (or benign) versus precancerous versus cancerous versus invasive or metastatic.

Applied to cytological specimens of cancer as a non-limiting exemplary example, the present invention provides a significant advance in providing diagnostically relevant information equivalent to that previously only available by histological sampling. In the case of breast cancer, cytology alone cannot differentiate cells of a specimen that are between the stages of ADH, DCIS, and IDC, for example. Standard cytology would classify such cells as “a typical” or “suspicious” which often leads to the requirement for an invasive surgical procedure to obtain a histological sample. For example, standard cytology cannot differentiate between

(i) normal (or benign) versus ADH cells (which is necessary to determine whether the cells are precancerous);

(ii) ADH versus DCIS or IDC cells, especially ADH versus low grade DCIS (which is necessary to determine whether the cells are cancerous); or

(iii) DCIS versus IDC (low or high grade) cells (which is necessary to determine whether the cells are invasive).

The above limitations also necessarily mean that standard cytology cannot differentiate between various grades or subtypes of cancerous cells.

Given these limitations, the utility of cytological specimens to assist in the diagnosis and treatment of patients, which are linked to known solid histology sample based diagnosis (like ADH, DCIS and IDC), is severely limited.

The present invention provides an advance in the utility of breast cytological specimens by first identifying “reference” molecular signatures of solid histological samples of diseased breast cells that have been correlated with specific stages and subtypes of breast cancer as phenotypes. A cytological sample from a patient is then obtained and cells isolated therefrom to determine, by comparison to “reference” molecular signatures, whether they are cancerous and/or which, if any, stage or subtype they reflect. For example, cells that are identified only as “a typical” by standard cytology may be isolated by microdissection and profiled by preparation of a molecular signature. The signature is then compared to “reference” signatures to determine whether the cells are ADH or DCIS

Generally, “reference” histological signatures of the invention are identified by analysis of biomolecule expression in multiple samples of each cancer type, stage or subtype to be studied. The overall gene expression profile of each sample is obtained by analyzing the expressed or unexpressed state of various genes (in the form of biomolecules expressed by said genes) in each cancer type, stage or subtype relative to each other (one gene to another across all genes). This overall profile is then analyzed to identify biomolecules, the expression or non-expression of which are positively, or negatively, correlated, with a type, stage or subtype of cancer relative to other biomolecules. A signature of a subset of biomolecules may then be identified by the methods of the present invention as correlated with particular cancer types, stages or subtypes. The use of multiple samples increases the confidence with which the expression status of a biomolecule is believed to be sufficiently correlated to a particular type, stage or subtype of cancer to contribute to a molecular model defining the cancer or its stages and subtypes. Without sufficient confidence, it remains unclear whether expression of a particular biomolecule is actually correlated with a type, stage or subtype of cancer and thus uncertain whether expression of a particular biomolecule may be successfully used to identify the type, stage or subtype of cells from a cytological specimen.

The “reference” molecular signatures of histological samples of a disease constitute a “molecular histological database” which includes information on identified gene expression patterns that discriminate between various types, stages, and subtypes of cancer. Such a database may be in electronic form, and may be accessed electronically.

In the case of breast cancer, the database would include signatures that provide information on one or more of items (1) through (7) as listed above. Such signatures may be that of measurements of particular protein levels, DNA levels, RNA levels and/or activity levels that can discriminate between various types, stages, and subtypes.

Moreover, the database may include “reference” signatures of cancer subtypes as they correlate with response to treatments such as surgery, radiation, and various therapeutic protocols (including, but not limited to, sensitivity or resistance to an anticancer agent, biotherapy, and small molecules) or combinations thereof as well as expected survival times for subjects afflicted with cancer cells displaying particular signatures. The correlation is provided by information on treatment regimen and outcomes in subjects from which “reference” signatures are obtained. The outcomes may be viewed as phenotypes that are observed in subjects afflicted with a particular stage (e.g. ADH, DCIS or IDC in breast cancer) or subtype (e.g. low versus high grade DCIS in breast cancer) of cancer as well as the results from therapies used to treat the cancer. “Reference” signatures that are correlated with one outcome versus another may be identified and used to identify a cell of a cytological specimen as being of one disease subtype rather than another. The levels or activities of one or more biomolecules that are assayed directly or indirectly in cells isolated from a cytological specimen are used to prepare a signature for comparison to “reference” signatures. The presence of a particular signature indicates the presence of cells corresponding to a particular subtype.

Methods for collecting cytological specimens for use in the present invention are known in the art and have been described herein. In the case of breast cancer, such specimens include fine needle aspirates and ductal lavage which can be used to prepare cytological smears or a ThinPrep®. These may then be reviewed by a cytologist or image analysis to identify cells of interest for which additional information is desirable. Cells of this type are typically those identified as a typical, suspicious or malignant, although benign cells may also be isolated for subtype analysis.

As an optional embodiment of the invention, the specimens are treated with a reagent that identifies cells of interest without the need for review by a cytologist. An example of this embodiment is the use of immunochemical analysis for cyclin D1, which has been observed as being found in a typical and DCIS cells but not benign lesions (see Oyama et al. Virchows Arch 435:413-421 (1999)). Use of such reagents readily supports automation of the present invention by permitting automated or partially automated selection of cells in a cytological specimen for isolation and preparation of a molecular signature. A reagent like that of an antibody for cyclin DI which is also observed in invasive cancer cells (in addition to a typical and DCIS cells) may also be used. Alternatively, a reagent that identifies cancerous cells (including invasive) but not normal, benign, or a typical cells would may also be used such that signatures can be prepared for comparison to “reference” signatures correlated to particular disease outcomes. Non-limiting examples include life expectancy and sensitivity or resistance to particular therapies.

The cells of interest are then isolated, preferably by microdissection, and used to prepare a molecular signature for comparison with “reference” histological signatures. A match with a “reference” signature permits a diagnosis of the cells as being a particular type, stage, and/or subtype of breast cancer.

The present invention may also be advantageously used where there are very few cells available in a cytological specimen. The ability to obtain and utilize a molecular signature from even a single cell for correlation with a phenotype permits the ability to successfully utilize a cytological specimen for diagnostic and prognostic purposes without the need for additional procedures (such as an invasive core biopsy) to obtain more cells.

Definitions of Terms as Used Herein:

A “molecular signature” or gene expression “pattern” or “profile” or variants of these terms refer to the relative expression of one or more biomolecules. In one aspect of the invention, the relative expression of biomolecules is between two or more types, stages and/or subtypes of disease which is correlated with being able to distinguish between said types, stages and/or subtypes. Each molecular signature thus corresponds to a phenotype to the exclusion of one or more other phenotypes. In preferred embodiments of the invention, expression of a biomolecule is detected by determining expression of a gene encoding said biomolecule or encoding a product affecting the presence or activity of a biomolecule. Alternatively, the amount or activity of a biomolecule may be assayed directly or indirectly as an indicator of its expression.

A “biomolecule” is any molecule that is made or utilized by a cell. The term includes, but it not limited to, nucleic acid (polynucleotide) molecules, polypeptide molecules, carbohydrate molecules, lipid molecules, and combinations thereof. The term also encompasses metabolites that are made or used by a cell, including small organic molecules.

A “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “stage” or “stages” (or equivalents thereof) of cancer refer to a physiologic state of a cell as defined by known histological (including immunohistology, histochemistry, and immunohistochemistry) procedures and are readily known to one skilled in the art. Non-limiting examples include normal versus abnormal, non-cancerous versus cancerous, the different stages described herein (e.g. hyperplastic, carcinoma, and invasive), and grades within different stages (e.g. grades I, II, or III or the equivalents thereof within cancerous stages).

The terms “correlate” or “correlation” or equivalents thereof refer to an association between expression of one or more genes and a physiologic state of a cell to the exclusion of one or more other type, stage, and/or subtype of a disease. The terms also refer to associations identified by use of the methods as described herein. A biomolecule or gene may be expressed at higher or lower levels and still be correlated with one or more cancer types, stages or subtypes.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence.

By corresponding is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17). Methods for amplifying mRNA are generally known in the art, and include reverse transcription PCR (RT-PCR); those described in U.S. Pat. Nos. 5,545,522, 5,716,785 and 5,891,636; and those described in U.S. patent application Ser. No. ______ (number to be assigned) entitled “Nucleic Acid Amplification” filed on Oct. 25, 2001 as attorney docket number 485772002900 as well as U.S. Provisional Patent Application No. 60/298,847 (filed Jun. 15, 2001), No. 60/257,801 (filed Dec. 22, 2000) and No. 60/364,492, filed Mar. 15, 2002, all of which are hereby incorporated by reference in their entireties as if fully set forth. Alternatively, RNA may be directly labeled as the corresponding cDNA by methods known in the art.

A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². Preferably, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of oligonucleotides or polynucleotides in the array is known, the identities of a sample polynucleotides can be determined based on their binding to a particular position in the microarray.

Because the invention relies upon the identification of biomolecules genes that may be over- or under-expressed, one embodiment of the invention involves determining expression by hybridization of mRNA, or an amplified or cloned version thereof, of a sample cell to a polynucleotide that is unique to a particular gene sequence. Preferred polynucleotides of this type contain at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Even more preferred are polynucleotides of at least about 25, at least about 50 or 60, at least about 100, and at least about 150 basepairs of a gene sequence that is not found in other gene sequences. The term “about” as used in the preceding sentence refers to an increase or decrease of 10% from the stated numerical value.

Alternatively, and in another embodiment of the invention, biomolecule or gene expression may be determined by analysis of expressed protein in a cell sample of interest by use of one or more antibodies specific for one or more epitopes of individual gene products (proteins) in said cell sample. Such antibodies are preferably labeled to permit their easy detection after binding to the gene product.

The term “label” refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

As used herein, a “cytological specimen” refers to a specimen of cells or cell containing fluid isolated from an individual suspected of being afflicted with, or at risk of developing, cancer. Cytological samples or specimens are of two basic types. The first utilizes either spontaneous or abraded (forcibly removed) exfoliates. Examples of the former are nipple secretions, vaginal fluids, cerebrospinal fluid, urine, or serrous effusions. Examples of the latter are ductal lavage, cervical smears, or other washings or brushings. The second type of cytological specimen is obtained by fine needle aspiration (FNA) biopsy. Such specimens are primary isolates (in contrast to cultured cells) and may be viewed as being collected through non-invasive or minimally invasive techniques which are readily performed in a clinical setting by use of devices and methods such as that described in U.S. Pat. No. 6,328,709, or any other suitable means recognized in the art. Such methods remove the cells from the tissue architecture in which they normally reside.

“Expression” and “gene expression” include transcription and/or translation of nucleic acid material.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions for production of a molecular signature. Other conditions include those used to prepare a cytological specimen for subsequent identification (e.g. staining) and isolation of one or more cells. These conditions also depend on what event is desired, such as hybridization, strand extension or transcription. For example, and in one embodiment, the methods of the present invention are allowed to be practiced under conditions where a skilled artisan is permitted to considered information in addition to the molecular signature of a cytological specimen to aid in the identification of the phenotype of the specimen. Examples of such information include whether the subject is at risk for a disease phenotype, has been previously diagnosed with a disease phenotype, has a disease phenotype in other tissues, or has other indicia of a disease phenotype.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion or insertion. Single nucleotide polymorphism (SNP) is also a sequence mutation as used herein. Because the present invention is based on the relative level of gene expression, mutations in non-coding regions of genes as disclosed herein may also be assayed in the practice of the invention.

“Detection” includes any means of detecting, including direct and indirect detection of gene expression and changes therein. For example, “detectably less” products may be observed directly or indirectly, and the term indicates any reduction (including the absence of detectable signal). Similarly, “detectably more” product means any increase, whether observed directly or indirectly.

The staining of cells as discussed herein may be performed by histochemical and immunochemical methods known in the art. These include staining with hematoxylin and eosin (H&E) or the PAP stain methods as well as the use of antibodies.

Unless defined otherwise all technical and scientific terms used herein have the same meaning as commonly understood to one of ordinary skill in the art to which this invention belongs.

Specific Embodiments

The present invention relates to the identification and use of molecular signatures (gene expression patterns or profiles) which discriminate between (or are correlated with) cells of various phenotypes, such as stages or subtypes of disease. The invention is particularly advantageously practiced in uses relating to cancer, but may also be practiced in cases of viral infections, where a cytological specimen is used to isolate cells suspected of being infected and then used to prepare a molecular signature for comparison to “reference” histological signatures of infected cells.

In preferred embodiments of the invention, the isolation of cells is by the use of laser capture microdissection (LCM) which advantageously permits the preparation of homogeneous cell populations from a cytological specimen. LCM has been predominantly used for preparative or sorting purposes rather than as an optical selection tool for analytical applications.

As applied to cancers, “reference” histological signatures may be determined by the methods of the invention by use of a number of reference histological samples that have been reviewed by a pathologist of ordinary skill and identified and/or diagnosed as being in the pathology of a given cancer. These reviewed samples include tissue architecture and thus “in situ” context for the identification/diagnosis. Signatures correlating with some subtypes may be identified by a further use of identified cancer stages in comparison to outcomes of the subjects from which the samples were obtained. Because the overall molecular signature differs from person to person, cancer to cancer, and cancer cell to cancer cell, correlations between certain cell states and biomolecules expressed or underexpressed may be made as disclosed herein to identify those that are capable of discriminating between different cancer types, stages and/or subtypes.

The present invention may be practiced with any number of biomolecules believed, or likely to be, differentially expressed in a phenotype, such as cancer. In one embodiment of the invention, the signature of a given stage and/or subtype of cancer is determined by using approximately 10,000 to 20,000 biomolecule encoding genes to identify hundreds of genes capable of discriminating between various the stages and/or subtypes of the cancer. For the identification of cancer types, especially cancers that may have an origin distinct from the location from which the cytological specimen was isolated, more genes may be used. The identification may be made by using gene expression profiles of various homogenous normal and cancer cell populations from histological samples, which were isolated by microdissection, such as, but not limited to, laser capture microdissection (LCM) of 100-1000 cells. Each gene of the expression profile may be assigned weights based on its ability to discriminate between two or more stages or subtypes of cancer. The magnitude of each assigned weight indicates the extent of difference in expression between the groups and is an approximation of the ability of expression of the gene to discriminate between the groups (and thus stages or subtypes). The magnitude of each assigned weight also approximates the extent of correlation between expression of individual gene(s) and particular cancer stages or subtypes.

It should be noted that merely high levels of expression in cells of a particular stage or subtype does not necessarily mean that a biomolecule or gene will be identified as having a high absolute weight value.

Genes with top ranking weights (in absolute terms) may be used to generate models of gene expression that would maximally discriminate between the groups. Alternatively, genes with top ranking weights (in absolute terms) may be used in combination with genes with lower weights without significant loss of ability to discriminate between groups. Such models may be generated by any appropriate means recognized in the art, including, but not limited to, cluster analysis, supported vector machines, neural networks or other algorithm known in the art. The models are capable of predicting the classification of a unknown cytological specimen based upon the expression of the genes used for discrimination in the models. “Leave one out” cross-validation may be used to test the performance of various models and to help identify weights (genes) that are uninformative or detrimental to the predictive ability of the models. Cross-validation may also be used to identify genes that enhance the predictive ability of the models.

The gene(s) identified as correlated with particular cancer stages or subtypes by the above models provide the ability to focus gene expression analysis to only those genes that contribute to the ability to identify a cell as being in a particular stage of cancer relative to another stage or subtype. The expression of other genes in a cancer cell would be relatively unable to provide information concerning, and thus assist in the discrimination of, different stages or subtypes of a cancer.

As will be appreciated by those skilled in the art, the models are highly useful with even a small set of reference gene expression data and can become increasingly accurate with the inclusion of more reference data although the incremental increase in accuracy will likely diminish with each additional datum. The preparation of additional reference gene expression data using genes identified and disclosed herein for discriminating between different stages or subtypes of cancer is routine and may be readily performed by the skilled artisan to permit the generation of models as described above to predict the status of an unknown cytological specimen based upon the expression levels of those genes.

To determine the expression levels of genes in the practice of the present invention, any method known in the art may be utilized. In one preferred embodiment of the invention, expression based on detection of RNA which hybridizes to the genes identified and disclosed herein is used. This is readily performed by any RNA detection or amplification+detection method known or recognized as equivalent in the art such as, but not limited to, reverse transcription-PCR, the methods disclosed in U.S. patent application Ser. No. ______ (number to be assigned) entitled “Nucleic Acid Amplification” filed on Oct. 25, 2001 as attorney docket number 485772002900 as well as U.S. Provisional Patent Application No. 60/298,847 (filed Jun. 15, 2001) and No. 60/257,801 (filed Dec. 22, 2000), and methods to detect the presence, or absence, of RNA stabilizing or destabilizing sequences.

Alternatively, expression based on detection of DNA status may be used. Detection of the DNA of an identified gene as methylated or deleted may be used for genes that have decreased expression in correlation with a particular breast cancer stage. This may be readily performed by PCR based methods known in the art. Conversely, detection of the DNA of an identified gene as amplified may be used for genes that have increased expression in correlation with a particular breast cancer stage. This may be readily performed by PCR based, fluorescent in situ hybridization (FISH) and chromosome in situ hybridization (CISH) methods known in the art.

Expression based on detection of a presence, increase, or decrease in protein levels or activity may also be used. Detection may be performed by any immunohistochemistry (IHC) based, blood based (especially for secreted proteins), antibody (including autoantibodies against the protein) based, exfoliate cell (from the cancer) based, mass spectroscopy based (e.g. Matrix Assisted Laser Desorption Ionization—Time Of Flight or MALDI-TOF), protein microarrays, and image (including used of labeled ligand) based method known in the art and recognized as appropriate for the detection of the protein. In one embodiment of the invention, IHC may be applied to a cytological specimen to detect expression of a biomolecule capable of discriminating between cancer stages and/or subtypes. Antibody and image based methods are additionally useful for the localization of tumors after determination of cancer by use of cells obtained by a non-invasive procedure (such as ductal lavage or fine needle aspiration), where the source of the cancerous cells is not known. A labeled antibody, substrate or ligand which binds to a biomolecule expressed in the cells may be used to localize the carcinoma(s) within a patient. In addition to applications in breast imaging, this embodiment of the invention may be used as part of any known imaging method (e.g. MRI, radiological, PET, etc.) by optionally labeling the antibody, substrate or ligand.

A preferred embodiment using a nucleic acid based assay to determine expression is by immobilization of one or more of the genes identified herein on a solid support, including, but not limited to, a solid substrate as an array or to beads or bead based technology as known in the art. Alternatively, solution based expression assays known in the art may also be used. The immobilized gene(s) may be in the form of polynucleotides that are unique or otherwise specific to the gene(s) such that the polynucleotide would be capable of hybridizing to a DNA or RNA corresponding to the gene(s). These polynucleotides may be the full length of the gene(s) or be short sequences of the genes that are optionally minimally interrupted (such as by mismatches or inserted non-complementary basepairs) such that hybridization with a DNA or RNA corresponding to the gene(s) is not affected.

The immobilized gene(s) may be used to determine the state of nucleic acid samples prepared from cell(s) of a cytological specimen for which the pre-cancer or cancer status is not known or for confirmation of a status that is already assigned to the cell(s). Without limiting the invention, such a specimen may be from a patient suspected of being afflicted with, or at risk of developing, a particular cancer known in the art to be possible in the tissue from which the specimen is prepared. The immobilized polynucleotide(s) need only be sufficient to specifically hybridize to the corresponding nucleic acid molecules derived from the cell(s) of the specimen. While even a single correlated gene sequence may to able to provide adequate accuracy in discriminating between two cancer cell stages or subtypes, two or more, three or more, four or more, five or more, six or more, seven or more, eight or more, nine or more, ten or more, twenty or more, fifty or more, one hundred or more, two hundred or more, five hundred or more, or one thousand or more of the genes may be used in combination to increase the accuracy of the method.

In embodiments where only one or a few genes are to be analyzed, the nucleic acid derived from the cell(s) of a specimen may be preferentially amplified by use of appropriate primers such that only the genes to be analyzed are amplified to reduce contaminating background signals from other genes expressed in the cell(s). Alternatively, and where multiple genes are to be analyzed or where very few cells (or one cell) is used, the nucleic acid from the cell(s) may be globally amplified before hybridization to the immobilized polynucleotides. Of course RNA, or the cDNA counterpart thereof may be directly labeled and used, without amplification, by methods known in the art.

The above assay embodiments may be used in a number of different ways to identify or detect the cancer stage or subtype, if any, of a cytological specimen from a patient. In many cases, this would reflect a secondary screen for the patient, who may have already undergone mammography or physical exam as a primary screen. If positive, a subsequent cytological specimen may be collected for use in the above assay embodiments.

The present invention provides a more objective set of criteria, in the form of gene expression profiles of a discrete set of genes, to discriminate (or delineate) between meaningful stages (or classes) or subtypes of cancer cells in a cytological specimen. In particularly preferred embodiments of the invention, the assays are used to discriminate between non-malignant and malignant cells, which is a critical determination for decisions concerning subsequent treatment and therapy for the patient. Another particularly preferred determination is between the three grades (I, II, III) of carcinomas in situ as well as the discrimination between grade III carcinomas in situ and invasive carcinomas. Other pairwise comparisons that are provided by the invention include, but are not limited to, normal versus cancerous (i.e. carcinoma present) and carcinoma in situ versus invasive. With the use of alternative algorithms, such as neural networks, comparisons that discriminate between multiple (more than pairwise) classes may also be performed. It is believed by the inventors that the present invention is the first example of objective, molecular criteria for making these discriminations in cytological specimens.

In an alternative embodiment of the invention, the cytological specimen of breast cancer may permit the collection of both normal and a typical cells for analysis. The gene expression patterns for each of these two cell types will be compared to each other as well as the model and the normal versus individual abnormal comparisons therein based upon the reference data set. This approach can be significantly more powerful than the a typical cells only approach because it utilizes significantly more information from the normal cells and the differences between normal and a typical cells (in both the sample and reference data sets) to determine the status of the a typical cells from the specimen.

By appropriate selection of the genes used in the analysis, identification of the relative amounts of cells in different stages of cancer may also be possible, although in most clinical settings, the identification of the highest grade of cancer with confidence makes identification of lower grades less important. Stated differently, the identification of invasive cancer determines the clinical situation regardless of the presence of carcinoma in situ or hyperplastic cells, or the identification of carcinoma in situ makes determines the clinical situation regardless of the presence of hyperplastic cells.

With use of the present invention, skilled physicians may prescribe treatments based on non-invasive cytological specimens which treatments were previously reserved for patients who had previously received a diagnosis via a solid tissue biopsy.

The above discussion is also applicable where a palpable lesion is detected followed by collection of a cytological specimen from the lesion. The cells are plated and reviewed by a pathologist or automated imaging system which selects cells for analysis as described above. This again provides a means of linking molecular cytology and molecular histology and provides an improved means of identifying the physiological state of breast cancer cells without the need for invasive solid tissue biopsies.

In a further alternative to all of the above, the gene(s) encoding biomolecule(s) identified herein may be used as part of a simple PCR or array based assay simply to determine the presence of a typical cells in a sample from a non-invasive sampling procedure. This is simple to perform and utilizes genes identified to be the best discriminators of normal versus abnormal cells without the need for any cytological examination. If no a typical cells are identified, no cytological examination is necessary. If a typical cells are identified, cytological examination follows, and a more comprehensive analysis, as described above, may follow.

The genes or biomolecules identified herein may be used to generate a model capable of predicting the cancer stage (if any) of an unknown cytological specimen based on the expression of the identified genes or biomolecules in the specimen. Such a model may be generated by any of the algorithms described herein or otherwise known in the art as well as those recognized as equivalent in the art using gene(s) (and subsets thereof) disclosed herein for the identification of whether an unknown or suspicious breast cancer specimen is normal or is in one or more stages of breast cancer. The model provides a means for comparing expression profiles of gene(s) of the subset from the specimen against the profiles of reference data used to build the model. The model can compare the specimen profile against each of the reference profiles or against model defining delineations made based upon the reference profiles. Additionally, relative values from the specimen profile may be used in comparison with the model or reference profiles.

In a preferred embodiment of the invention, cells identified as normal and abnormal (a typical) from a cytological specimen from the same subject may be analyzed for their expression profiles of the genes used to generate the model. This provides an advantageous means of identifying the stage of the abnormal sample based on relative differences from the expression profile of the normal sample. These differences can then be used in comparison to differences between normal and individual abnormal reference data which was also used to generate the model.

The detection of gene expression from a cytological specimen may be by use of a single microarray able to assay gene expression from all pairwise comparisons disclosed herein for convenience and accuracy.

Other uses of the present invention include providing the ability to identify cancer cell samples as being those of a particular stage of cancer for further research or study. This provides a particular advantage in many contexts requiring the identification of cancer stage based on objective genetic or molecular criteria rather than cytological observation. It is of particular utility to distinguish different grades of a particular cancer stage for further study, research or characterization because no objective criteria for such delineation was previously available.

The materials for use in the methods of the present invention are ideally suited for preparation of kits produced in accordance with well known procedures. The invention thus provides kits comprising agents for the detection of expression of the disclosed genes for identifying breast cancer stage. Such kits optionally comprising the agent with an identifying description or label or instructions relating to their use in the methods of the present invention, is provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

The methods provided by the present invention may also be automated in whole or in part. All aspects of the present invention may also be practiced such that they consist essentially of a subset of the disclosed materials and processes to the exclusion of subject matter irrelevant to the detection of disease presence or identification of disease (cancer) stages and/or subtypes in a cytological specimen.

Having now generally described the invention, the same will be more readily understood through reference to the following examples which are provided by way of illustration, and are not intended to be limiting of the present invention, unless specified.

### EXAMPLES

### Example I

### Materials and Methods

Clinical Breast Cancer Specimens

Clinical biopsies from 30 patients were obtained from the Massachusetts General Hospital with Institutional Review Board approval. The tissue from one of the patients was not associated with breast cancer of any kind since it was from a breast reduction procedure. Pathological and histological information for the biopsies were also obtained. Three independent captures of about 1000 breast epithelial cells of one or more of the four different disease stages (normal, N; a typical ductal hyperplasia, ADH; ductal carcinoma in situ, DCIS; invasive ductal carcinoma, IDC) were procured from each biopsy using Laser Capture Microdissection (LCM, Arcturus Engineering). Three independent captures of LCIS (lobular carcinoma in situ) in one biopsy were also made. Total RNA was extracted from the captured (procured) cells and amplified with a T7-promoter based RNA amplification protocol. The human universal reference RNA (Stratagene, La Jolla), was similarly amplified and used as the reference channel in a two-color microarray hybridization.

Microarrays

To maximize coverage of breast cancer-related genes on the microarrays used, 11,435 cDNA clones from the IMAGE consortium (Research Genetics) were obtained. These clones were selected based on literature knowledge (such as, but not limited to, preferential expression in cancer versus normal cells) and after mining (such as, but not limited to, preferential expression in breast tissues) gene expression information in the expressed sequence tags (EST) databases and the Serial Analysis of Gene Expression (SAGE) data sets available from the National Center for Biotechnology Information (NCBI, http://www/ncbi.nlm.nih.gov).

Microarray Data Processing

Microarray images were analyzed with ImaGene (BioDiscovery) to find and quantitate each spot on the microarray. Spots flagged by ImaGene as poor spots using standard criteria used with the software for the standardization of signals were excluded from further analysis. Raw Cy5 (sample channel) and Cy3 (reference channel) intensities and associated local background estimates for each spot were then examined. The signal/noise ratio, defined as the spot intensity over background intensity, was used as the second criteria for spot exclusion; spots with signal/noise ratio <3.0 in the reference channel or <1.5 in the sample channel were excluded from further analysis. Background-subtracted intensities across the chip were normalized to the 75th-percentile of the spot intensity distribution on the entire chip (alternative normalizations to the mean, median or other point may also be used as known in the art). Cy5/Cy3 ratios of each spot for each cellular state were averaged across each of six measurements (3 LCM captures×2 chips/capture=6 chips); outliers among the 6 data points were removed before taking the average. The resulting data were formatted as a data matrix (samples along the top horizontal axis and gene identity along the vertical axis) for data mining (see FIG. 1 with data).

Microarray Data Analysis

Before further analysis, each value in a row (gene) of the gene expression matrix was divided by the median value for the row, and the resulting matrix log-transformed. Normalized, median-centered, and log-transformed, gene expression data matrix was loaded into GeneMaths software (Applied-Maths, Belgium). Clustering and discriminant analysis were performed to identify sets of genes associated with different cellular states. For each pair-wise comparison between two breast cancer stages, samples are assigned to either the positive group or negative group, and genes were sorted by their discriminatory weights. The absolute value of the weight of a gene indicates the extent of difference in expression between the two groups; the positively signed genes are expressed higher in one group and the negatively singed genes are expressed higher in the other group.

The utility of the top-ranking genes as a diagnostic test was evaluated using the support vector machines (SVMs) algorithm (see Yeang, C. H., S. Ramaswamy, et al. (2001). “Molecular classification of multiple tumor types.” Bioinformatics 17 Suppl 1: S316-22; Xiong, M., X. Fang, et al. (2001). “Biomarker identification by feature wrappers.” Genome Res 11(11): 1878-87; Furey, T. S., N. Cristianini, et al. (2000). “Support vector machine classification and validation of cancer tissue samples using microarray expression data.” Bioinformatics 16(10): 906-14; and Brown, M. P., W. N. Grundy, et al. (2000). “Knowledge-based analysis of microarray gene expression data by using support vector machines.” Proc Natl Acad Sci U S A 97(1): 262-7, who state “SVMs are considered a supervised computer learning method because they exploit prior knowledge of gene function to identify unknown genes of similar function from expression data. SVMs avoid several problems associated with unsupervised clustering methods, such as hierarchical clustering and self-organizing maps.”) Other algorithms, such as, but not limited to, linear discriminate analysis, logistic regression, cluster analysis, K-th nearest neighbor, or neural nets.

The support vector machines algorithm finds the maximal margin hyperplane that separate the two groups under comparison. The method of leave-one-out cross-validation was used to test the performance of a given set of genes; one sample was taken in turn out of the training set and a model is built using the rest of the training set, which is in then applied to classify the left-out sample. The accuracy of the genes in the cross-validation procedure is the percentage of correct classifications over the total number of the training samples.

“Reference” Molecular Signatures and “Reference” Database

A histological sample, such as solid tissue (e.g. from a biopsy or surgical resection) is obtained and prepared for histological analysis. Optional preparative methods may be any known in the art, including, but not limited to, formalin fixed paraffin embedded (FFPE) samples or frozen (e.g. −80° C.) samples that are sectioned and post-fixed (e.g. with ethanol, methanol, acetone, formalin). The sample is then stained and analyzed for cells of a particular disease, disease stage, disease subtype, or other phenotype. Cells of a particular phenotype are isolated by microdissection, such as laser capture microdissection.

Using breast cancer as an example, cells are stained (e.g. with hematoxylin and eosin (H&E)or an immunostain) and used to identify those of various phenotypes (e.g. benign; ADH; DCIS, I, II, III; and/or IDC, I, II, III). The cells are isolated according to phenotype. Stated differently, specific cells of different benign or pathological stages or states are captured. In the generation of “reference” signatures, larger numbers of cells, as well as samples from multiple patients, are preferred to reduce the effects of individual variations between cells and samples and permit the identification of signatures that correlate with all cells of a given phenotype.

The isolated cells are used to prepare nucleic acids or other cellular components reflective of the level of expression of biomolecules in the cells. The nucleic acids or other cellular components may be used directly, such as by hybridization to a nucleic acid containing microarray followed by analysis, or processed before further analysis. Examples of further processing include amplification, such as by quantitative PCR (Q-PCR) or amplification by any of the methods discussed herein. Prior amplification (e.g. with label introduced as part of the amplification process) is preferred before hybridization to a nucleic acid containing microarray. In an alternative embodiment, amplification (e.g. QPCR) may be used after hybridization to a microarray. In an additional alternative, nucleic acids may be amplified by one method and then subjected to QPCR.

The biomolecule expression levels are molecular signatures that are compiled into a reference database with identification of the phenotype of the cells from which the signature was prepared. The database is compared to molecular signatures from cytological specimens to identify the specimen as having the same signature, and thus phenotype, as cells from a histological sample. Individual reference signatures identified with the same phenotype may also be compared and/or combined such that variations in signatures due to different cells or different samples are excluded from the reference signature. This is also referred to as building a “model” reference signature of a phenotype that is used for comparisons with signatures from cytological specimens to identify the specimen as having the same signature, and thus phenotype, as cells from the histological samples used to build the “model” signature.

In an alternative embodiment, the “model” signature may be based on a comparison and/or combination of reference signatures from more than one phenotype. For example, reference signatures from various subtypes of benign phenotypes may be combined to produce a “model” benign signature. In another embodiment, reference signatures from various subtypes of malignant phenotypes may be combined to produce a “model” malignant signature.

### Example II

### Cytological Specimens from Breast Cancer

As noted above, a variety of non-invasive means may be use to prepare cytological specimens. For breast cancer, non-limiting examples include fine needle aspiration (FNA), nipple aspirate, or ductal lavage as well as scrapings of ulcerated lesions and nipple secretions. These initial isolates may be processed by any means known in the art, including the concentration of cells therein followed by immobilization of the cells on a slide suitable for staining and subsequent microdissection.

For example, the initial isolate may be processed by with the collection of cells by centrifugation followed by resuspension (optionally in a preservative solution) in a small volume. The cells may then be collected on a membranous filter for subsequent transfer to a thin preparation like that on a slide, which allows the specimen to be stained (optionally with the stain for PAP smears) and used for the isolation of cells by microdissection.

In addition to the discussion above concerning application of the present invention to identify cells of a breast cytological specimen as having signatures corresponding to various normal and diseased phenotypes, a specimen may also be analyzed for signatures corresponding to conditions such as duct adenocarcinoma NOS (not otherwise specified), lobular carcinoma, medullary carcinoma, mucinous (colloid) carcinoma, apocrine carcinoma, papillary carcinoma, papilloma, and tubular carcinoma as well as metastatic carcinoma, secretory carcinoma, and signet ring carcinoma. Preferably, the invention is applied with respect to particular pathological stages of breast cancer (e.g. ADH, LG-DCIS, HG-DCIS, LG-DCIS, LG-IDC, and HG-IDC) and/or the pathological grades (i.e. grades I, II, and III) of DCIS and IDC.

The invention is also advantageously applied in situations of desmoplastic tumors that yield few cells of a malignant lobular carcinoma for cytological analysis and of lobular carcinomas in general because of their diffuse infiltrative pattern makes differentiation from benign duct cells difficult.

The present invention is also applied to the differentiation of nonproliferative breast disease, such as fibrocystic change (including cystic change with florid apocrine metaplasia), adenosis and radial scar from conditions such as apocrine carcinoma, galactocele, traumatic fat necrosis, intraductal papilloma, intracystic papillary carcinoma, cystic necrosis of duct carcinoma, adenocarcinoma (NOS with cystic change or comedocarcinoma), metaplastic carcinoma, and/or inflammatory breast lesions such as acute mastitis breast abscess, chronic subareolar abscess, and the aforementioned comedocarcinoma and fat necrosis. In another embodiment, the present invention is applied to the differentiation of proliferative breast disease, which includes a typical hyperplasia, from ductal/lobular carcinoma in situ and fibroadenoma, tubular carcinoma, and/or invasive carcinoma.

The present invention is also applied to the differentiation of pregnancy and lactation induced changes, such as lactating adenoma, tubular adenoma, or fibroadenoma with lactating change, from the above described conditions. Benign neoplasms of the breast, including intraductal papilloma and fibroadenoma, may also be differentiated by the present invention from the above conditions as well as phyllodes tumor, mesenchymal neoplasms, mucocele like tumor, and/or mucinous carcinoma.

The above noted conditions may be diagnosed or otherwise identified by histological and/or cytopathological/cytomorphological criteria such that known cell populations may be used to identify “reference” molecular signatures for use in the present invention.

### Example III

### Cytological Specimens from Squamous Lesions

Squamous lesions of the female reproductive tract (including the cervix and vagina) have been observed as having a wide range of cellular alterations, including benign changes and changes to preneoplastic or neoplastic states. Many of these have no clear relation to carcinogenesis, and have been grouped together as “a typical squamous cells or undetermined significance” or “ASCUS” as part of The Bethesda System (TBS) for cervical-vaginal cytology. The present invention may be applied to identify cells of a cytological specimen from the female reproductive tract as having signatures corresponding to various normal and diseased phenotypes.

TBS classification includes ASCUS, squamous intra epithelial lesions (SIL) and squamous carcinoma, with the first being the most unclear and often being defined in relation to the latter two. The grading of SIL includes the distinguishing of low grade lesions (mild dysplasia/cervical intraepithelial neoplasia, or CIN, Grade I) and high grade SIL (including moderate displasia/cervical intraepithelial neoplasia (CIN), grade II; severe displasia/carcinoma in situ of the non-keratinizing type and CIN, grade III; and severe keratinizing displasia) by both histological and cytopathological features. The present invention is applied such that “reference” molecular signatures of each of these conditions are identified and used in comparison to molecular signatures of cells present in a cytological specimen. The present invention is also advantageously applied in situations of small numbers of dysplastic squamous cells, where the ability to obtain a molecular signature from only a few cells provides for diagnosis unavailable by cytology alone.

SIL may also have the involvement of human papilloma virus (HPV) which results in different histological and cytopathological features. The present invention is applied to utilize molecular signatures correlating to HPV involved SIL to identify cells of a cytological specimen as having the same phenotype. This may be practice by using signatures of HPV encoded biomolecules and/or signatures of cellular biomolecules that are altered in expression due to HPV infection.

High grade SIL (HSIL) also presents the need to differentiate carcinoma in situ of the nonkeratinizing type from discrete severely dysplastic/in situ cells such as immature squamous metaplastic cells, endometrial stromal cells (superficial and deep), and intrauterine device associated changes in cells. HSIL also needs to be differentiated from tissue fragments of severely dysplastic/in situ cells such as tissue fragments of squamous metaplasia, tissue fragments of atrophic parabasal cells, tubal metaplasia, microglandular hyperplasia, endocervical adenocarcinoma in situ, and endometrial adenocarcinoma. The present invention is applied to utilize molecular signatures that correlate cells of a cytological specimen with HISL rather than these other diagnostic entities that complicate the diagnosis. Of course the present invention may also be used to identify molecular signatures corresponding to these other conditions as well.

In a similar manner, molecular signatures corresponding to keratinizing high grade SIL as opposed to diagnostic entities such as hyperkeratosis/parakeratosis, cellular changes in the background of atrophy, HPV associated changes, and well differentiated (keratinizing) squamous cell carcinoma are identified and used to diagnose cells of a cytological specimen as having the same phenotype. Molecular signatures corresponding to microinvasive or superficially invasive squamous carcinoma may also be identified and used. In the latter group, molecular signatures corresponding to well differentiated (keratinizing) squamous cell carcinoma and/or moderately to poorly differentiated squamous carcinoma, as well as stages and subtypes thereof, may also be identified and used.

Molecular signatures of small cell undifferentiated or neuroendocrine carcinoma of the cervix may also be identified and used. Moreover, and with respect to ASCUS, the invention may be used to identify molecular signatures for use in differentiating cells of this classification apart from any of the above as well as into the phenotypes of changes involving mature squamous cells; changes involving intermediate type squamous cells (with low grade SIL, or LSIL, inflammation associated changes, radiation and chemotherapy induced changes, decidual W cells, and atrophy associated changes as non-limiting examples); and changes involving parabasal and metaplastic cells, such as excessively keratinized pleomorphic squamous cells.

All of the above noted conditions may be diagnosed or otherwise identified by histological and/or cytopathological/cytomorphological criteria such that known cell populations may be used to identify “reference” molecular signatures for use in the present invention.

In addition to reducing the risk of false negatives in gynecological cytopathology, this application of the invention also permits improvements in performing a differential diagnosis with respect to additional conditions such as, but not limited to, cells undergoing repair/regeneration, squamous cells in atrophy, pemphigus vulgaris, and metastatic adenocarcinoma.

### Example IV

### Cytological Specimens from the Endometrium and Endocervix

In addition to cervical cancer, endometrial adenocarcinoma is an ongoing malignant neoplasm of the female genital tract. This adenocarcinoma is rarely detected in cervical-vaginal smears, although direct sampling by endometrial washings or aspirations offers additional means for its detection. Detection of endometrial lesions by analysis of cervical-vagina smears poses diagnostic problems such as differentiating endometrial cells of a benign phenotype (including hyperplasia) from those of benign appearing low grade adenocarcinoma; identifying endometrial carcinomas in samples with few carcinoma cells, especially where the subject is asymptomatic or high risk; recognizing nonspecific cytopathological features that co-exist with low grade endometrial carcinomas as a clue to malignancy when they are found alone; and distinguishing poorly differentiated endometrial carcinomas from conditions such as endocervical adenocarcinoma, squamous cell carcinoma, and metastatic carcinoma. The present invention may be applied to identify cells of an endometrial or endocervical cytological specimen as having signatures corresponding to various normal and diseased phenotypes.

Contributing to the above problems are the presence of endometrial metaplasia and hyperplasia in contrast to adenocarcinoma of the endometrium, which is classified as typical endometrial or endometroid adenocarcinoma with or without squamous differentiation; villoglandular; adenosquamous carcinoma; mucinous adenocarcinoma; serous adenocarcinoma; clear cell carcinoma; squamous cell carcinoma; undifferentiated carcinoma; mixed types of carcinomas, miscellaneous types of carcinomas; and metastatic carcinoma. Another type of endometrial carcinoma is uterine papillary serous carcinoma. The present invention is applied to identify and use molecular signatures correlating to these classifications as well as subtypes thereof to identify cells of a cytological specimen as having the same phenotype (or subtype thereof).

The present invention is also applied advantageously to differentiate the above from the abnormal presence of endometrial cells (during the latter half of the mentrual cycle or in postmenopausal women due to chronic endometritis, IUD usage, dysfunctional uterine bleeding, pregnancy, abortion related procedures, postpartum effects, endometriosis of the cervix or vagina, sampling of uterine tissue by endocervical sampling, withdrawal bleeding in women with hormonal replacement therapy, endometrial polyp whether hyperplastic or atrophic, endometrial surface metaplasia, and/or endometrial hyperplasia).

Additional examples of the present invention applied to differential diagnosis of endometroid or endometrial carcinomas include identifying and using molecular signatures correlating to benign endometrial cells (including chronic endometritis; dysfunctional uterine bleeding; endometrial polyps; uterine leiomyomas; endometrial surface epithelial metaplasia, papillary syncytial type; endometrial hyperplasia, lower uterine segment cells, and cervical/vaginal endometriosis); squamous carcinoma in situ; poorly differentiated squamous cell carcinoma with small cell pattern; and/or neuroendocrine carcinoma.

The present invention is also applied to identifying and using molecular signatures correlating to uterine sarcomas, such as leiomyosarcomas, endometrial stromal sarcomas, and malignant mixed mesodermal tumors (MMMT) as well as subtypes thereof.

Gynecological cytopathology also poses the situation of endocervical related diseases, which requires differentiation of a number of classifications of endocervix conditions from each other and other conditions. Non-limiting examples of such classification include a typical glandular cells of undetermined significance (AGUS); endocervical adenocarcinoma (in situ and invasive); benign endocervical glandular changes (including microglandular hyperplasia of the cervix and tubal metaplasia); endometrial and metastatic carcinomas; and poorly differentiated squamous cell carcinoma. The present invention is applied to identifying in situ and invasive endocervical adenocarcinomas from each other; differentiating benign changes from malignant ones; identifying AGUS as cells of another classification or subtypes within AGUS; identifying endocervical adenocarcinoma from endometrial and metastatic carcinomas and poorly differentiated squamous cell carcinoma; and/or identifying squamous cell carcinoma in situ from endocervical adenocarcinoma, whether in situ or invasive.

The present invention is also applied to identifying various adenocarcinomas of the uterine cervix (both in situ and invasive forms) from each other and from other conditions (such as tubal metaplasia, microglandular hyperplasia, endocervical gladular atypia, and squamous carcinoma in situ). Molecular signatures of endocervical adenocarcinoma in situ (including typical endocervical adenocarcinoma; endometroid adenocarcinoma; clear cell adenocarcinoma, tubulocystic and papillary; papillary mucinous adenocarcinoma; papillary serous adenocarcinoma; mucinous, colloid, intestinal adenocarcinoma; medullary adenocarcinoma; adenoid cystic carcinoma; adenosquamous carcinoma; adenoma malignum) are identified and used in comparison to molecular signatures of cells present in a cytological specimen.

All of the above noted conditions may be diagnosed or otherwise identified by histological and/or cytopathological/cytomorphological criteria such that known cell populations may be used to identify “reference” molecular signatures for use in the present invention. This application of the invention also permits improvements in performing a differential diagnosis with respect to additional conditions such as, but not limited to, cells undergoing repair/regeneration, effects from endogenous or exogenous progesterone (e.g. pregnancy or hormonal therapy), endometrial adenocarcinoma, nonkeratinizing squamous cell carcinoma, and extrauterine adenocarcinoma.

### Example V

### Cytological Specimens from Serous (Body) Cavities

Serous effusions, as well as pelvic/peritoneal washings and cul-de-sac fluids, are analyzed cytologically to detect the presence or absence of malignancy, although the presence of malignant cells in such fluids indicates advanced disease. The present invention is applied to identifying reactive/hyperplastic mesothelial cells from malignant mesothelioma or metastatic adenocarcinoma; identifying malignant mesothelioma from metastatic adenocarcinoma or other malignancies; improving analysis of effusions with large numbers of lymphoid cells; and identifying primary sites of malignancy in malignant effusions without a known source of malignancy. The present invention may be applied to identify cells of a cytological specimen from serous cavities as having signatures corresponding to various normal and diseased phenotypes.

The histology and cytology of normal or benign mesothelial cells, including reactive or hyperplastic mesothelial cells, are known and are used to identify “reference” molecular signatures. Signatures of reactive mesothelial cells may be combined with signatures identified from malignant mesothelioma, malignant lymphoma, malignant mesothelioma adenocarcinoma, squamous cell carcinoma, malignant melanoma (including amelanotic), (poorly differentiated) adenocarcinoma, megakaryotes, rheumatoid pleuritis, granulomatous inflammation, granulosa cell tumor, and small cell undifferentiated carcinoma to permit their use with molecular signatures of cytological specimens of serous effusions.

Additionally, the present invention is used to differentiate cytological specimens of reactive mesothelial hyperplasia, metastatic adenocarcinoma, papillary adenocarcinoma (including that of the lung, ovary, thyroid, kidney and serous papillary tumor of the peritoneum), squamous cell carcinoma, malignant melanoma, and metastatic cancers from each other by molecular signatures relative to “reference” signatures.

All of the above noted conditions may be diagnosed or otherwise identified by histological and/or cytopathological/cytomorphological criteria relative to the source of cells found in serous cavities such that known cell populations may be used to identify “reference” molecular signatures for use in the present invention.

### Example VI

### Cytological Specimens from the Respiratory Tract (Exfoliative and Aspiration)

Specimens from the respiratory tract include sputum; bronchial brushings or washings; bronchoalveolar lavage; tracheal aspiration; and percutaneous transthoracic or transbronchial FNA. These are usually used in relation to detecting lung cancer. The present invention may be applied to identify cells of a cytological specimen from the respiratory tract as having signatures corresponding to various normal and diseased phenotypes.

In one embodiment, the present invention is applied to detecting lung cancer despite different cytomorphology depending on specimen type; contamination with large numbers of respiratory epithelial cells and/or mesothelial tissue fragments; a typical squamous cells in transbronchial aspiration of patients treated with radiation; squamous cells that overlie submucosal cancers; and different morphological patterns seen with lung cancer; reducing the reliance on the presence of lymphocytes in lymph nodes to stage lung cancers. The invention is also applied to identifying squamous cell carcinoma from reactive/recovering or metaplastic squamous cells and radiation/chemotherapy induced changes; identifying adenocarcinoma from hyperplastic bronchial epithelial cells and a typical type II pneumocytes; identifying small cell undifferentiated carcinoma from folliculat bronchitis and reserve cell hyperplasia; identifying reactive mesothelium from adenocarcinoma or malignant mesothelioma; identifying malignant neoplasms with cavitation from cavitary infectious lesions; typing primary versus metastatic carcinoma; typing bronchogenic adenocarcinoma versus bronchioloalveolar adenocarcinoma; and/or differentiation of neuroendocrine tumors, small cell neoplasms, and/or malignant mesothelioma. The invention is also applied to discriminating between major types of lung cancer, including squamous cell carcinoma, brochogenic adenocarcinoma, bronchioloalveolar carcinoma, small cell undifferentiated carcinoma, and/or large cell undifferentiated carcinoma, which are all histologically identifiable. All of the above may be performed by correlating molecular signatures of cytological specimens with those of histological samples.

With respect to squamous cell carcinoma, the present invention is also applied to discrimination, by use of molecular signatures, of squamous metaplasia with dysplasia, squamous carcinoma in situ, squamous cell carcinoma, repair/recovering cells, cells subjected to radiation and chemotherapy, and vegetable cells. With respect to bronchogenic and bronchioloalveolar adenocarcinomas, the present invention uses molecular signatures for discrimination of them from brochial epithelial hyperplasia, type II pneumocytes hypertrophy/hyperplasia, and/or reactive bronchial epithelial cells. Furthermore, the present invention is used to differentiate cells with foamy cytoplasm from reactive type II pneumocytes, lipid pneumonia, goblet cell metaplasia, and/or primary and secondary mucin producing adenocarcinoma.

With respect to small cell undifferentiated carcinomas, the present invention uses molecular signatures for discrimination of them from reserve cell hyperplasia, follicular bronchitis, malignant lymphoma, carcinoid (grade I neuroendocrine carcinoma), bronchial cell hyperplasia, poorly differentiated carcinoma, adenoid cystic carcinoma, alveolar lining cells, and/or malignant melanoma. With respect to pulmonary neuroendocrine tumors, the present invention is also applied to discrimination, by use of molecular signatures, of carcinoid tumor (grade I neuroendocrine carcinoma, excluding spindle cell variant), a typical carcinoid (grade II neuroendocrine carcinoma), small cell undifferentiated carcinoma (grade III neuroendocrine carcinoma), and large cell neuroendocrine carcinoma (grade III neuroendocrine carcinoma) from each other and from other conditions. Among grade I neuroendocrine carcinoma (a type of carcinoid tumor), the invention is applied to discriminating against reserve cell hyperplasia, bronchial epithelial cell hyperplasia, alveolar lining cells, lymphocytes, malignant lymphoma, adenoid cystic carcinoma, poorly differentiated squamous cell carcinoma, and/or primary and metastatic adenocarcinoma. Among carcinoid tumors of the spindle type, the present invention is applied to discriminating against spindle cell thymoma, soft tissue tumors, malignant melanoma, and/or medullary thyroid carcinoma.

Additionally, this aspect of the invention includes the identification and use of molecular signatures corresponding to phenotypes of viral infections of the respiratory tract for correlations with molecular signatures of cell(s) from a cytological specimen of a subject.

All of the above noted conditions may be diagnosed or otherwise identified by histological and/or cytopathological/cytomorphological criteria such that known cell populations may be used to identify “reference” molecular signatures for use in the present invention.

### Example VII

### Cytological Specimens from the Alimentary Tract (Esophagus/Gastrointestinal and Biliary Tracts)

Despite the ability to obtain tissue biopsies, gastrointestinal cytology continues to be performed on esophageal, gastric, and duodenal brushings, colonic and rectal brushings, brushings of the ampulla of Vater, common bile duct, and pancreatic duct, fluid from the pancreatic duct, and bile specimens. The present invention may be applied to identify cells of a cytological specimen from the alimentary tract as having signatures corresponding to various normal and diseased phenotypes.

In the esophagus, the present invention is applied to confirm a diagnosis of Barrett's esophagus, monitor for neoplasms following abnormal results, and monitor treatments of esophageal carcinoma by radiation or other regimens. Therefore, the invention includes the use of molecular signatures of the phenotypes of normal or benign cells (such as reflux esophagitis or radiation induced changes), Barrett's esophagus cells, malignant neoplasms of the esophagus (such as squamous cell carcinoma, adenocarcinoma, and/or small cell undifferentiated carcinoma) for correlation with molecular signatures of cytological specimens.

With respect to the stomach, the present invention includes differentiation of repair/recovery changes from carcinoma, of signet ring cell carcinoma from malignant lymphoma, and/or of histiocytes from individually dispersed malignant cells (and from gastritis or gastric ulcers). This aspect includes the discrimination of normal gastric mucosa from non-neoplastic lesions of the stomach from malignant neoplasms of the stomach (including adenocarcinoma, whether of the intestinal or diffuse/gastric type) from malignant lymphoma from neuroendocrine tumors from smooth muscle tumors.

With respect to bile and biliary tract specimens, the present invention includes differentiation of benign/reactive cells from well differentiated adenocarcinoma and/or poorly differentiated carcinoma.

All of the above noted conditions may be diagnosed or otherwise identified by histological and/or cytopathological/cytomorphological criteria such that known cell populations may be used to identify “reference” molecular signatures for use in the present invention.

### Example VIII

### Cytological Specimens from the Urinary Tract

The present invention may be applied to identify cells of a cytological specimen from the urinary tract as having signatures corresponding to various normal and diseased phenotypes. Urinary cytology has a number of limitations to which the present invention may be applied. Specifically, the present invention utilizes molecular signatures to differentiate benign transitional epithelium from low grade urothelial neoplasms; transitional (dysplasia) cells due to intraepithelial abnormalities from low grade neoplasms (papilloma or grade I as well as grade II types); low grade neoplasms from transitional cell carcinoma in situ (TIS) and grade III transitional cell carcinoma from low grade neoplasms; and/or TIS from invasive high grade transitional cell carcinoma.

Urinary bladder cancer is due to epithelial, urothelial or transitional cell carcinoma; squamous cell carcinoma; and adenocarcinoma, all of which may be identified by molecular signatures used in the practice of the invention. Signatures of transitional cell carcinomas classified as being low grade (grades I and II) and high grade (grades II and III as well as TIS) may also be used in the practice of the invention. Similarly, the invention may be applied to differentiating between reactive (or regenerative/recovering) urothelium, dysplasia, and/or the low grades of transitional cell carcinoma.

Other neoplasms that may be differentially identified from the above by the present invention are neuroendocrine carcinomas, sarcomas of the urinary bladder, metastatic carcinomas (e.g. from the prostate, colon-rectum, uterine, cervix, and endometrium), malignant lymphomas, melanomas, and/or renal cell carcinomas. The present invention may also be applied to differentiating the above from nephrogenic adenoma, cellular changes due to topical chemotherapy (e.g. treatment with thiotepa, mitomycin, or Bacillus Calmette-Guerin (BCG) vaccine), cellular changes due to systemic chemotherapy (e.g. cyclophosphamide), radiation induced changes in cells, and/or virus induced changes in cells (e.g. infection by human polyomavirus). The last of these is particularly important to differentiate from high grade transitional cell carcinoma.

All of the above noted conditions may be diagnosed or otherwise identified by histological and/or cytopathological/cytomorphological criteria such that known cell populations may be used to identify “reference” molecular signatures for use in the present invention.

### Example IX

### Cytological Specimens from the Thyroid

The present invention may be applied to identify cells of a cytological specimen from the thyroid as having signatures corresponding to various normal and diseased phenotypes. Thyroid nodules are common, although mostly benign. Fine needle biopsy (aspiration) is used to provide distinctions between benign and/or malignant lesions because it is not possible to do so clinically, via radionuclide imaging, or by ultrasonography. The present invention is most advantageously applied to difficulties in aspiration cytopathology of the thyroid such as 1) differentiating non-neoplastic (e.g. nodular goiter or chronic lymphocytic thyroiditis) from neoplastic diseases (e.g. benign and malignant neoplasms); 2) differentiating benign thyroid neoplasms (e.g. follicular adenoma and Hurthle cell adenoma) from malignant neoplasms (e.g. follicular, papillary, or medullary carcinoma and Hurthle cell carcinoma); 3) differentiating types of malignant neoplasms (e.g. papillary carcinoma from follicular carcinoma, and medullary carcinoma from Hurthle cell, anaplastic, papillary, or follicular carcinoma); and/or 4) differentiating primary thyroid cancer from metastatic malignancy to the thyroid.

Applications 1) and 2) are particularly significant because they have therapeutic implications with respect to the use of subsequent surgical procedures. Specifically, application 1) may be used to differentiate nodular goiter from follicular neoplasm (adenoma/carcinoma), papillary carcinoma, Hurthle cell neoplasm, anaplastic carcinoma, medullary carcinoma, and/or metastatic carcinoma. The benign neoplasm follicular adenoma may also be differentiated into colloid or macrofollicular adenoma, simple or normofollicular, microfollicular, and/or trabecular by use of the present invention. The malignant neoplasm follicular carcinoma may be differentiated from hyperplastic nodular goiter, follicular nodule in Hashimoto's Thyroiditis (chronic lymphocytic thyroiditis), microfollicular and trabecular types of follicular adenoma, and/or follicular variant of papillary carcinoma. Nodular goiter with cystic change may also be differentiated from cystic papillary carcinoma.

Application 2) may be used to differentiate papillary thyroid carcinoma (PTC) from hyperplastic goiter; papillary change in follicular nodules or follicular adenoma; Hashimoto's Thyroiditis; follicular adenoma; follicular hyperplasia in Hashimoto's Thyroiditis; hyperplastic goiter or nodular goiter with degeneration and cyst formation; hyalinizing trabecular adenoma; nodular goiter, and/or parathyroid hyperplasia/adenoma. Another use is to differentiate papillary carcinoma from papillary hyperplasia (papillary change in nodular goiter), follicular adenoma, hyalinizing trabecular adenoma, and/or papillary hyperplasia in Hashimoto's Thyroiditis. Yet a further use is to differentiate medullary carcinoma from nodular and amyloid goiter.

Application 3) may be used to differentiate poorly differentiated “insular” carcinoma from malignant lymphomas, medullary carcinomas with small cell pattern; anaplastic carcinoma from medullary carcinoma, metastatic poorly differentiated malignant neoplasm, malignant lymphoma and malignant melanoma; and/or medullary carcinoma from Hurthle cell carcinoma, papillary carcinoma (single cell pattern), follicular neoplasms (e.g. hyalinizing trabecular adenoma, cellular follicular adenoma, and follicular carcinoma), and/or anaplastic carcinoma. Application 3) may also be modified to differentiate PTC into the following variants: usual or conventional type; follicular variant; tall cell variant; columnar cell variant; oxyphilic variant; solid and trabecular variant; diffuse sclerosing; papillary carcinoma with nodular fascitis like stroma; macrofollicular; diffuse follicular; papillary microcarcinoma; and/or encapsulated.

The invention may also be used to differentiate malignant lymphoma from Hashimoto's Thyroiditis Lymphoid Type, Hashimoto's Thyroiditis with a typical lymphoid hyperplasia, metastatic small cell undifferentiated carcinoma, and/or anaplastic carcinoma.

All of the above noted conditions may be diagnosed or otherwise identified by histological and/or cytopathological/cytomorphological criteria such that known cell populations may be used to identify “reference” molecular signatures for use in the present invention.

### Example X

### Cytological Specimens from Lymph Nodes

The present invention may be applied to identify cells of a cytological specimen from a lymph node as having signatures corresponding to various normal and diseased phenotypes. Fine needle aspiration biopsy is used in evaluation of lymphadenopathies. The present invention is most advantageously applied to difficulties in aspiration cytology of lymph nodes such as 1) differentiating malignant lymphomas from reactive processes; 2) differentiating some types of malignant lymphomas from epithelial and mesenchymal malignancies; 3) diagnosing the present of small cell lymphomas; 4) subclassification of malignant lymphoma; and/or 5) reducing the occurrence of false negatives in desmoplastic stroma, as in cases of primary mediastinal lymphomas or nodular sclerosing Hodgkin's disease. The invention may also be used to classify non-neoplastic lymphadenopathies as idiopathic reactive hyperplasia (follicular pattern), virus induced (e.g. infection by mononucleosis, cytomegalovirus, and HIV), florid follicular hyperplasia, dermatopathic lymphadenitis, lymphadenopathy due to Dilantin, sacoidosis, mycobacterial infection, fungal infection (e.g. Histoplasma or Cryptococcus), cat scratch disease, or toxoplasma.

Another application is the differentiation of malignant lymphoma as non-Hodgkin's lymphoma or Hodgkin's lymphoma as well as the classification of the former as diffuse small lymphocytic, small lymphocytic with plasmacytoid features, mantle cell lymphoma, small cleaved cell, small non-cleaved cell (Burkitt's and non-Burkitt's types), lymphoblastic lymphoma (convoluted cell and non-convoluted cell), mixed small and large cleaved cell, peripheral T-cell lymphoma, large cleaved and non-cleaved, large non-cleaved and immunoblastic, or anaplastic Ki-1 lymphoma. Hodgkin's lymphoma may also be differentiated from thymoma, germ cell tumors, reactive lymphadenopathy (virus induced), extramedullary hematopoiesis, and myelolipoma (all of which may be considered lymphocyte predominant or mixed cell) as well as from peripheral T-cell lymphoma, poorly differentiated carcinoma, soft tissue sarcoma with pleomorphic pattern, malignant melanoma, and/or anaplastic large cell or Ki-1 lymphoma (all of which may be considered lymphocyte depleted).

The present invention may also be applied to differentiated the above from plasmacytoma, granulocytic sarcoma, Langerhans cell Histiocytosis, malignant histiocytosis, and metastatic tumors such as squamous carcinoma, adenocarcinoma, neuroendocrine tumors, malignant melanoma, and/or soft tissue tumors.

Many of the above noted conditions may be diagnosed or otherwise identified by histological and/or cytopathological/cytomorphological criteria relative to the source of cells in lymph nodes such that known cell populations may be used to identify “reference” molecular signatures for use in the present invention.

### Example XI

### Cytological Specimens from Salivary Glands

The present invention may be applied to identify cells of a cytological specimen from a salivary gland as having signatures corresponding to various normal and diseased phenotypes. Fine needle aspiration biopsy is used in evaluation of salivary gland lesions. The present invention is most advantageously applied to the differentiation of the large number of benign and malignant neoplasms involving the salivary glands. The present invention may be applied to the differentiation among benign and malignant conditions such as pleomorphic adenoma, Warthin's tumor, bronchial cleft cyst, adenoid cystic carcinoma, mucoepidermoid carcinoma (low, intermediate, and high grade), chronic sialadenitis, monomorphic adenoma, low grade adenocarcinoma, polymorphous low grade adenocarcinoma, soft tissue tumor, oncocytoma, adenoid cystic carcinoma, basal cell carcinoma, benign lymphoepithelial cyst, benign lymphoepithelial lesion, intraparotid lymph node with reactive features, malignant non-Hodgkin's lymphoma (primary or secondary), mucocele, pleomorphic adenoma, low grade mucoepidermoid carcinoma, mucus retention cyst, dermoid and epidermoid cysts, cystic metastasis of keratinizing squamous cell carcinoma, and papillary oncocytic cystadenoma. Low grade mucoepidermoid carcinoma may also be differentiated from mucocele and necrotizing sialometaplasia.

The present invention may also be applied to the differentiation of acinic cell carcinoma from low grade adenocarcinoma, poorly differentiated adenocarcinoma NOS (not otherwise specified), polymorphous low grade adenocarcinoma, pleomorphic adenoma with predominant or exclusive epithelial component, salivary duct carcinoma, high grade mucoepidermoid carcinoma, and/or metastatic malignancies such as poorly differentiated squamous carcinoma/adenocarcinoma or amelanotic malignant melanoma. Alternatively, the invention may be applied to the differentiation of small cell neoplasms from basal cell adenoma, basal cell adenocarcinoma, adenoid cystic carcinoma, small cell undifferentiated carcinoma (primary or secondary), malignant lymphoma, poorly differentiated squamous carcinoma, and/or pleomorphic adenoma with predominant or exclusive basaloid cell pattern.

Another set of differential diagnosis that may be performed by application of the present invention is among forms of clear cell neoplasms (oncocytomas with clear cell change, mucoepidermoid carcinoma, clear cell carcinoma, epithelial-myoepithelial carcinoma, acinic cell carcinoma, and metastatic renal cell carcinoma) as well as apart from spindle cell neoplasms, myoepithelioma or pleomorphic adenoma with an extensive myoepithelial component, fibrous histiocytoma, and/or Schwannoma nerve sheath tumor.

Many of the above noted conditions may be diagnosed or otherwise identified by histological and/or cytopathological/cytomorphological criteria relative to the source of cells in salivary glands such that known cell populations may be used to identify “reference” molecular signatures for use in the present invention.

### Example XII

### Cytological Specimens from Liver

The present invention may be applied to identify cells of a cytological specimen from liver as having signatures corresponding to various normal and diseased phenotypes. Aspiration biopsy is used in evaluation of liver lesions. The present invention is most advantageously applied to the differentiation of reactive/regenerative hepatocytes from hepatocellular carcinoma; differentiation between focal nodular hyperplasia, hepatocellular adenoma, and hepatocellular carcinoma; and/or differentiation between hepatocellular carcinoma and other malignancies. Examples of reactive/regenerative (recovery) changes of the liver include fatty changes, inflammatory conditions hepatic abscess, hepatic cyst, hydatid cyst, and focal nodular hyperplasia. The present invention may be applied to the differentiation of the above from primary hepatic neoplasms including hemangiomas, hepatocellular adenoma, hepatocellular carcinoma, cholangiocarcinoma, angiosarcoma, and/or metastatic tumors including adenocarcinoma, squamous cell carcinoma, malignant melanoma, renal cell carcinoma, neoplasms of the thyroid, leimyocarcoma, and neuroendocrine tumors. Hepatoblastoma may also be differentiated from the above by use of the present invention.

All of the above noted conditions may be diagnosed or otherwise identified by histological and/or cytopathological/cytomorphological criteria such that known cell populations may be used to identify “reference” molecular signatures for use in the present invention.

### Example XIII

### Cytological Specimens from Pancreas

The present invention may be applied to identify cells of a cytological specimen from pancreas as having signatures corresponding to various normal and diseased phenotypes. Fine needle aspiration biopsy is used in evaluation of pancreas to detect mass lesions. The present invention is most advantageously applied to the identification of pancreatic neoplasms as arising from other sources, such as exocrine cells (e.g. benign and malignant cystic neoplasms of duct epithelium, duct adenocarcinoma, and acinar cell carcinoma) and neuroendocrine cells (e.g. islet cell tumors); differentiating chronic pancreatitis with fibrosis (with or without florid ductal hyperplasia with or without nuclear atypia) from well differentiated ductal adenocarcinoma; differentiation of well differentiated ductal adenocarcinoma with small cell pattern from acinar cell carcinoma, neuroendocrine tumors (islet cell tumor), and solid and cystic tumor of the pancreas (papillary-cystic tumor); differentiating cystic carcinomas from benign neoplastic cysts and pseudocysts; and/or acinar cell carcinomas from benign acinar tissue.

The present invention may also be applied to the differentiation of the above from serous cystadenoma (microcystic adenoma), mucinous neoplasms (mucinous cystadenoma and cystadenocarcinoma), and/or duct adenocarcinoma with cystic change.

All of the above noted conditions may be diagnosed or otherwise identified by histological and/or cytopathological/cytomorphological criteria such that known cell populations may be used to identify “reference” molecular signatures for use in the present invention.

### Example XIV

### Cytological Specimens from Kidney

The present invention may be applied to identify cells of a cytological specimen from kidney as having signatures corresponding to various normal and diseased phenotypes. While radiographic studies are used to diagnose renal cell carcinomas, fine needle aspiration biopsy is used with cystic lesions with radiographic or clinical suspicion of malignancy; to provide a morphological diagnosis prior to radiation or chemotherapy of an advanced malignancy; to confirm a metastatic malignancy; to confirm a malignant diagnosis in a patient for whom surgical intervention is not possible; and to confirm a recurrence of renal carcinoma after nephrectomy. The present invention is most advantageously applied to the differentiation of cystic renal cell carcinomas with poor cellularity from benign renal cysts and cystic nephroma; differentiation of renal cell carcinoma of clear type from adrenal cortical cells, cortical hyperplasia or adenoma; differentiation of renal oncocytoma from renal cell carcinoma (conventional and chromophobe types); differentiation of renal cortical papillary adenoma from renal cell carcinoma; differentiation of renal tubular cells from low grade renal cell carcinoma; differentiation of hepatocytes from renal cell carcinoma; differentiation of poorly differentiated or high grade renal cell carcinoma from metastatic poorly differentiated carcinomas; differentiation of low grade transitional cell carcinoma of renal pelvis/ureters from benign urothelium; and/or differentiation of high grade transitional cell carcinoma from poorly differentiated renal cell carcinoma.

The present invention may be applied to the differentiation cystic lesions of the kidney from cystic carcinoma and from cystic nephroma (or multilocular cyst of the kidney), a benign neoplasm. Other benign neoplasms of the kidney that may be differentiated by use of the present invention include papillary adenoma (which may be differentiated from papillary carcinoma by the present invention), renal oncocytoma (which may be differentiated from classic or conventional renal cell carcinoma with a predominant granular cell component and/or oncocytic variant of chromophobe renal cell carcinoma by the present invention), and/or renal angiomyolipoma.

The present invention may also be used to differentiate the above from renal cell carcinoma, including clear cell type (classic or conventional), chromophobe type, papillary carcinoma, oncocytoma, collecting duct carcinoma, and/or epithelioid angiomyolipoma. Low grade renal cell carcinomas may also be differentiated from normal tubular cells, renal abscess/pyelonephritis, xanthogranulomatous pyelonephritis, adrenal cortical cells or cortical hyperplasia/adenoma, and/or renal infarct. Primary renal carcinoma can also be differentiated from metastatic neoplasms, such as malignant lymphoma. Unclassified renal cell carcinoma can also be differentiated into various phenotypes by the present invention.

All of the above noted conditions may be diagnosed or otherwise identified by histological and/or cytopathological/cytomorphological criteria such that known cell populations may be used to identify “reference” molecular signatures for use in the present invention.

### Example XV

### Application of the Invention to Phenotypes Corresponding to Outcomes

In all of the above Examples, the present invention may be applied by use of “reference” molecular signatures that identify of disease subtypes based upon disease outcomes (or “outcome phenotypes”). This may be done by identifying signatures in histological samples before and after a period of time wherein the disease is treated or not treated. For example, various histological samples with a particular disease phenotype may be obtained from multiple subjects and used to prepare molecular signatures that are correlated with the disease outcome in said subjects. Possible outcomes include those from the lack of treatment (e.g. life expectancy), as well as with treatment of various modalities, including surgical, different radiation, and different chemotherapeutic regimens. The outcomes of treatment also include the sensitivity or resistance of the disease phenotype to the treatment. Molecular signatures can also be prepared from samples obtained after the various treatments for comparison to the signatures found prior to treatment to identify additional disease phenotypes based upon the signatures. Alternatively, molecular signatures are obtained from subjects after treatment and correlated with outcomes that have occurred in said subjects.

Outcome phenotypes may be used in comparison to molecular signatures of a cytological specimen from a subject to identify said specimen as having the phenotype and to assist the skilled practitioner in selecting a treatment for the subject and/or advising the subject on the likely prognosis of the disease.

### Example XVI

### Automation of the Invention

The present invention may be automated in whole or in part as discussed above and as computer assisted electronic analyses. Reference signatures identified by use of the present invention may be stored electronically in a computer or electronically used thereby to generate a model signature for a particular phenotype. The molecular signature of a cytological specimen may then be compared to reference signatures and/or a model generated therewith electronically. The identification of the signature of a cytological specimen may be performed electronically (e.g. by a computer), especially where identification consists of analyzing an array (e.g. microarray) containing information relating to the expression of biomolecules which make up the signature. A computer then compares the signature of the cytological specimen to one or more reference signatures, or models generated therewith, to identify the specimen as having a particular phenotype relative to a histological sample. Where reference signatures correlated with outcomes are used, the computer may also identify possible treatment regimens for the subject from whom the specimen was taken.

### REFERENCES

DeRisi, J., et al., Use of a cDNA microarray to analyse gene expression patterns in human cancer, Nature Genetics, (1996) 14:457-460.

Hedenfalk, I., et al., Gene-Expression Profiles In Heredity Breast Cancer, The New England Journal of Medicine, (Feb. 22, 2001) 344:8:539-548.

Golub, T. R., et al., Molecular Classification of Cancer: Class Discovery and Class Prediction by Gene Expression Monitoring, Science, (Oct. 15, 1999) 286:531-537.

Perou, Charles M., et al., Molecular portraits of human breast tumours, Nature, (Aug. 17, 2000) 406:747-752.

Garber, Mitchell E., et al., Diversity of gene expression in adenocarcinoma of the lung, Proc. Natl. Acad. Sci. USA, (Nov. 20, 2001) 98:24:13784-13789.

Perou, Charles M., et al., Distinctive gene expression patterns in human mammary epithelial cells and breast cancers, Proc. Natl. Acad. Sci. USA, (August 1999) 96:9212-9217.

Sgrio, Dennis C., et al., In Vivo Gene Expression Profile Analysis of Human Breast Cancer Progression, Cancer Research, (Nov. 15, 1999) 59:5656-5661.

Sorlie, Therese, et al., Gene expression patterns of breast carcinomas distinguish tumor subclasses with clinical implications, Proc. Natl. Acad. Sci., (Sep. 11, 2001) 98:19:10869-10874.

Alizadeh, Ash A., et al., Distinct types of diffuse large B-cell lymphoma identified by gene expression profiling, Nature, (Feb. 3, 2000) 403:503-511.

Bittner, M., et al., Molecular classification of cutaneous malignant melanoma by gene expression profiling, Nature (Aug. 3, 2000) 406:536-540.

West, M., et al., Predicting the clinical status of human breast cancer by using gene expression profiles, Proc. Natl. Acad. Sci., (Sep. 25, 2001) 98:20:11462-11467.

Kini, S. R. Color Atlas of Differential Diagnosis in Exfoliative and Aspiration Cytopathology. Philadelphia: Lippincott Williams & Wilkins, 1999.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

We claim:

1. A method of correlating a molecular signature of cell(s) of a cytological specimen with the phenotype of cell(s) of a histological sample comprising
obtaining and preparing a cell containing cytological specimen from a subject;
isolating one or more cells from said specimen;
preparing a molecular signature from said one or more cells wherein said signature is reflective of the levels or activities of one or more biomolecules in the isolated cell(s); and
comparing said molecular signature to a molecular signature reflective of the levels or activities of biomolecules in cells of a histological sample wherein a positive correlation between the signatures indicates the cell(s) of the specimen as having the phenotype of the sample.

2. The method of claim 1 wherein said molecular signature comprises expression of more than one gene.

3. The method of claim 2 wherein said molecular signature is embodied on a microarray.

4. The method of claim 1 wherein said phenotype is the presence of a malignant disease.

5. The method of claim 1 wherein said phenotype is a stage of a benign or malignant disease.

6. The method of claim 1 wherein said phenotype defines a subtype of a disease indicative of responsiveness to a therapeutic treatment.

7. The method of claim 1 wherein said subject is a member of the population at large or is suspected of being afflicted with a disease.

8. The method of claim 1 wherein said disease is cancer.

9. The method of claim 8 wherein the disease is cancer of the breast, colon, pancreas, liver, salivary glands, lymph nodes, thyroid, urinary bladder, lung, cervix, or endometrium.

10. The method of claim 9 wherein the disease is breast cancer.

11. The method of claim 1 wherein said specimen is obtained by ductal lavage or fine needle aspiration

12. The method of claim 1 wherein said isolating is by microdissection.

13. The method of claim 1 wherein said molecular signature is the expression level of mRNA transcripts.

14. A method for determining the presence of a disease in a subject comprising:
obtaining and preparing a cell containing cytological specimen from said subject;
isolating one or more cells suspected of being indicative of said disease from said specimen;
measuring the levels or activities of one or more biomolecules in the isolated cells; and
comparing said levels or activities to the levels or activities of said biomolecules in cells of a histological sample identified as having said disease.

15. The method of claim 14 wherein said disease is cancer.

16. The method of claim 14 wherein the disease is cancer of the breast, colon, pancreas, liver, salivary glands, lymph nodes, thyroid, urinary bladder, lung, cervix, or endometrium.

17. The method of claim 16 wherein the disease is breast cancer.

18. The method of claim 14 wherein said isolating is microdissection.

19. The method of claim 14 wherein said measuring is by determining the expression level of mRNA transcripts

20. A method for determining whether a cytological specimen contains benign or malignant cancer cells comprising:
obtaining and preparing a cell containing cytological specimen from a subject;
isolating one or more cells within said specimen which may be either benign or malignant;
measuring the levels or activities of one or more biomolecules in the isolated cells; and
comparing said levels or activities to the levels or activities of said biomolecules in cells of different benign and/or malignant cancer cells of a histological sample.

21. The method of claim 20 wherein said specimen is from human breast, colon, pancreas, liver, salivary glands, lymph nodes, thyroid, urinary bladder, lung, cervix, or endometrium.

22. The method of claim 21 wherein said specimen is from human breast.

23. The method of claim 20 wherein said measuring is determining the expression level of said biomolecules.

24. The method of claim 23 wherein expression level of mRNA transcripts is determined.

25. The method of claim 20 wherein the biomolecules are RNA, DNA, and protein.

26. The method of claim 20 wherein said isolating is by laser capture microdissection.

27. A method of identifying molecular signatures that correlate a cytological specimen with the phenotype of a histological sample comprising
obtaining the molecular signatures of a plurality of histological samples of a single phenotype,
comparing the signatures to identify biomolecules the expression of which correlate with said phenotype,
and confirming the presence of said phenotype in cells of a cytological specimen.

28. A method of identifying biomolecules that discriminate between two or more stages or subtypes of cancer comprising
measuring the levels or activities of a plurality of biomolecules in cells isolated by microdissection from histological samples of a plurality of subjects wherein said cells are identified as being of particular stages or subtypes of cancer;
identifying biomolecules the levels or activities of which correlate with said particular stages or subtypes.

29. A method of identifying a cell of a cytological specimen as having the phenotype of a subtype of cancer comprising
obtaining and preparing a cell containing cytological specimen from a subject;
isolating one or more cells within said specimen;
preparing a molecular signature from said one or more cells wherein said signature is reflective of the levels or activities of one or more biomolecules in the isolated cell(s); and
comparing said molecular signature to a molecular signature of cells of a histological sample identified as that of a subtype of cancer wherein a positive correlation between the signatures indicates the cell(s) of the specimen as having the phenotype of the subtype.

30. The method of claim 29 wherein a subtype is defined by an outcome observed in subjects having cells with the molecular signature of said histological sample.

31. The method of claim 30 wherein said said subtype comprises sensitivity or resistance to an anticancer therapy and/or survival times of said subject.

32. An array comprising immobilized reagents complexed with biomolecules derived from a cytological specimen wherein said array represents a molecular signature of said specimen.

33. The array of claim 32 wherein said specimen is from a subject afflicted with, or suspected of afflicted with, cancer.

34. The array of claim 32 wherein said reagents are nucleic acid molecules.

35. The array of claim 32 wherein said biomolecules are nucleic acid molecules and are complexed with said reagents by hybridization.

36. The array of claim 35 wherein said biomolecules are mRNA or amplified from RNA molecules in said specimen.

37. The array of claim 33 wherein said cancer is breast cancer.

38. A method of identifying a molecular signature of a subset of a histological sample comprising
comparing reference molecular signatures of cells microdissected from histological samples of more than one subject identified as having the same phenotype;
identifying the expression of one or more biomolecules in more than one, but not all, of the reference signatures;
identifying said expression as the molecular signature of a subset of said histological sample.

39. The method of claim 38 wherein said phenotype is a disease phenotype.

40. The method of claim 38 further comprising correlating said molecular signature of a subset with a phenotype based upon comparison with observations at the cell, tissue, system, and/or organism level of the subjects.
